SEQUENCE LISTING (4 sequences)

SEQ ID NO: 1 - DNA, 32 bp, Homo sapiens
  source 1..32: mol_type = other DNA
ttttttggtggtggtggttgtggtggtggtgg

SEQ ID NO: 2 - DNA, 31 bp, Homo sapiens
  source 1..31: mol_type = other DNA
ttttttgcagttgatcctttggataccctgg

SEQ ID NO: 3 - DNA, 92 bp, Homo sapiens
  source 1..92: mol_type = other DNA
ttttttaaccgcccaaatccctaagagtctgcacttgtcattttgtatatgtatttggtttttggctctcacagacacactacacacgcaca

SEQ ID NO: 4 - DNA, 54 bp, Homo sapiens
  source 1..54: mol_type = other DNA
ttttttcactacagaggttgcgtctgtcccacgttgtcatggggggttggcctg

(19) United States
(12) Patent Application Publication
(10) Pub. No.: US 2023/0341376 A1
(43) Pub. Date: Oct. 26, 2023

(54) SYSTEM, METHOD, AND COMPUTER-ACCESSIBLE MEDIUM FOR PHENOTYPING OF SINGLE CELLS WITH MULTIPLEXED VIBRATIONAL PROBES

(71) Applicant: The Trustees of Columbia University in the City of New York, New York, NY (US)

(72) Inventors: Wei MIN, Fort Lee, NJ (US); Zhilun ZHAO, Guangdong (CN)

(21) Appl. No.: 17/958,005

(22) Filed: Sep. 30, 2022

Related U.S. Application Data
(63) Continuation of application No. PCT/US2021/025577, filed on Apr. 2, 2021.
(60) Provisional application No. 63/004,153, filed on Apr. 2, 2020.

(51) Int. Cl. G01N 33/50; G01N 15/14

(52) U.S. Cl. CPC G01N 33/5023; G01N 15/1434; G01N 15/1484; G01N 33/5011; G01N 2015/1488; G01N 2458/00

(57) ABSTRACT

An exemplar system, and computer-accessible medium for determining phenotypic (e.g., multi-parameter) information for a cell(s) can include, for example, generating spectral information of the cell(s) using a Raman spectroscopy procedure that is based on a vibrational probe(s), and determining the metabolic, protein biomarker, or signaling pathway information based on the spectral information. The Raman spectroscopy procedure can be a single-cell spontaneous Raman spectroscopy procedure. The single-cell Raman spectroscopy procedure can be performed using a whole-cell confocal micro-Raman spectrometer. The vibrational probe(s) can include a Deuterium-labeled branched-chain amino acid(s), or a deuterium-labeled palmitic acid(s), Raman active nanoparticles or other essential metabolites such as glucose and water.

### CROSS-REFERENCE TO RELATED APPLICATION(S)

This application is a continuation of International Patent Application No. PCT/US2021/025577, filed on Apr. 2, 2021 that published as International Patent Publication No. WO 2021/202997 on Oct. 7, 2021, and also relates to and claims priority from U.S. Pat. Application No. 63/004,153, filed on Apr. 2, 2020, the entire disclosures of which are incorporated herein by reference.

### STATEMENT REGARDING FEDERALLY SPONSORED RESEARCH

This invention was made with government support under Grant No. R01EB020892, awarded by the National Institutes of Health. The government has certain rights in the invention.

### SEQUENCE LISTING

The instant application contains a Sequence Listing XML, which has been submitted via EFS-Web and is hereby incorporated by reference in its entirety. The Sequence Listing XML, created on Jun. 9, 2023, is named 17958005-109198-284-seq.xml, and is 4,776 bytes in size.

### FIELD OF THE DISCLOSURE

The present disclosure relates generally to multiplexed vibrational probes, and more specifically, to exemplary embodiments of exemplary system, method, and computer-accessible medium for metabolic activity phenotyping of single cells with multiplexed vibrational probes.

### BACKGROUND INFORMATION

Genetics and metabolism are certain defining characteristics of life -- it is the synthesis, transformation and degradation of biomolecules (e.g., metabolism) inside each cell that carry out the genetic blueprint. Hence, quantitative measurements of the metabolism of individual cells are essential to understand questions in diverse fields in biology and medicine. (See, e.g., Reference 1). In cancer biology, for example, it is known that the metabolic profiles can be altered in many cancer cells and serve as druggable targets. (See, e.g., Reference 2). Such altered metabolism can vary from cell to cell within tumor tissue. Termed as intratumoral heterogeneity, this chaotic nature of metabolism in individual cells can lead to heterogeneous tumor growth, invasion and drug resistance. (See, e.g., Reference 3). Therefore, the task of evaluating single-cell metabolism is becoming increasingly urgent, especially in the post-genomics era. Similar tasks are also demanded in drug discovery, particularly for those drug targeting metabolic pathways, as the rapid determination of the mechanisms of compound candidates greatly facilitates drug screening. (See, e.g., References 4, 5). While recent development of multi-parameter profiling approaches has provided important insights into the mechanism of drug action, many of these analyses are not directly linked to the drug targets, limiting their interpretability. (See, e.g., References 6-9). Therefore, a method that quantitatively provides insights into the metabolism of single cells is in demand.

In addition to the metabolic measurements in single cells, it can be desirable to profile multiple biomarkers such as surface proteins as well as other pathways such as endocytosis, e.g., both at single cell level.

Among available approaches, mass-spectrometry is widely used for metabolic profiling due to its capability of evaluating a large pool of metabolites at the same time. Yet, mass-spectrometry can be rather challenging to resolve at single-cell level. Besides, mass-spectrometry is destructive, which makes it impossible to collect cells for additional downstream analysis. On the other hand, optical procedures can be advantageous for their non-destructive nature, easy sample preparation, and high throughput capability. (See, e.g., Reference 10). Fluorescence-based flow cytometry, for example, is routinely used for immune cell phenotyping, biomarker detection with great sensitivity and specificity. However, while good at profiling surface biomarkers, fluorescence methods may have, for example, limited utility in reporting metabolism, as the fluorescent probes are too bulky to label small metabolites. On the other hand, label-free Raman microscopy generally relies on the physical and chemical properties of cells to generate information about cell phenotypes. (See, e.g., References 11, 12). However, the physical and chemical information acquired by label-free Raman microscopy is difficult to explain any changes in metabolism or surface markers, which often hinders mechanistic insights for fundamental understanding.

Label-free methods, such as Raman-based vibrational microscopy, rely on physical and chemical properties to generate information about cell phenotypes (see, e.g., Reference 52), and usually don’t demand a priori knowledge of biomarkers. For this reason, label-free Raman spectroscopy has been extensively investigated and successfully demonstrated in many diagnostic applications. (See, e.g., References 53-55). In the past decade, great efforts have been put into improving the hardware in order to increase the signal to noise ratio, (see, e.g., References 56-58), and into developing sophisticated statistical tools such as machine learning procedures to achieve better diagnostic results. (See, e.g., Reference 59). Yet challenges remain that hinder the prevalence of this powerful procedure in broader applications. Among those challenges, to achieve a robust and reproducible detection and classification classifier model is the key.

Additionally, comprehensive understanding of fundamental biology, disease mechanisms, and drug therapeutics can require the integration of information from a large number of related pathways. Multiplexed measurements are thus attracting considerable interest in single-cell biology and systems biology. (See, e.g., References A1-A3). In the context of single-cell analysis, mass cytometry is popular for large-scale proteomics. (See, e.g., Reference A4). However, mass cytometry may be restricted by its destructive nature, and thus it is incompatible with the investigation of live cells and downstream cell sorting. In contrast, fluorescence-based flow cytometry can be an optical method that can interrogate live cells non-destructively. (See, e.g., References A5, A6). However, fluorescence detection may be limited by the broad fluorescent spectra, and the associated “color barrier” constraints the multiplexing level of fluorescence to just a few channels. Although, e.g., up to 17 parameters have been reported in the literature, the implementation of multiparameter fluorescence cytometry can be rather laborious and challenging due to cumbersome and error-prone spectral compensation and high instrument complexity of multiple lasers and detectors. (See, e.g., References A5, A7). Moreover, metabolites may be difficult for fluorescence detection because of the relatively bulky size of fluorophores compared to small metabolites. (See, e.g., References A8-A10), and as a result, the crucial functional information about metabolic activities can often be missing in fluorescence-based live-cell profiling. Furthermore, fluorescence detection often suffers from auto-fluorescence and photobleaching. (See, e.g., Reference All).

It is possible to exploit Raman scattering as an alternative solution to super-multiplex live-cell profiling. Raman scattering can be a powerful optical spectroscopy that can complement fluorescence in some aspects. First, in the condensed phase, e.g., Raman spectrum can typically be 50 times narrower than that of fluorescence. Thus, Raman spectroscopy can principally circumvent the “color barrier” of fluorescence and hold great promise for super-multiplexing. (See, e.g., References A12-A15). Technically, compared to multiple lasers and detectors required in fluorescence cytometry, the single laser (which excites all Raman modes) and single Raman detector (which collects all modes) configuration can permit robust readout of multiparameter data with the one-shot acquisition. Second, e.g., Raman spectroscopy may not require bulky fluorophores, making it potentially capable of detecting small metabolites. (See, e.g., Reference A16). Third, Raman spectroscopy can be free from photobleaching.

However, Raman-based live-cell profiling is still in its early stages. (See, e.g., References A17-A20). This is largely because functional Raman probes may be lagging behind the counterparts in mass cytometry or fluorescence flow cytometry. In particular, for profiling specific protein markers, compared to the well-established fluorophore-conjugated antibodies or rare-earth meta-isotope-tagged antibodies (see, e.g., References A5, A21-A24), the existing antibody-based Raman probes may be limited by insufficient brightness and/or low level of multiplexing, despite extensive efforts in developing organic dyes, (see, e.g., References A25, A26), polymer nanoparticles, (see, e.g., References A27, A28), and metallic nanoparticles, (see, e.g., References A29, A30). In a sense, Raman spectroscopy may not fulfill its full potential without, e.g., the use of matching functional probes.

Thus, it may be beneficial to provide an exemplary system, method, and computer-accessible medium for multi-parameter phenotyping of single cells with multiplexed vibrational probes which can overcome at least some of the deficiencies described herein above.

### SUMMARY OF EXEMPLARY EMBODIMENTS

An exemplary system, and computer-accessible medium for determining metabolic information for a cell(s) can include, for example, generating spectral information of the cell(s) using a Raman spectroscopy procedure that can be based on a profiling probe(s), and determining the metabolic, biomarker and signaling pathway information based on the spectral information. The Raman spectroscopy procedure can be a single-cell Raman spectroscopy procedure. The single-cell Raman spectroscopy procedure can be performed using a whole-cell confocal micro-Raman spectrometer. The whole-cell confocal micro-Raman spectrometer can have an illumination spot size of between about 1 µm to about 5 µm, between about 5 µm to about 10 µm, or between about 10 µm to about 100 µm. The whole-cell confocal micro-Raman spectrometer can have a pinhole size between about 50 µm to about 200 µ, between about 200 µm to about 400 µm, or between about 400 µm to about 1000 µm.

In certain exemplary embodiments of the present disclosure, the profiling probe(s) can include a Deuterium-labeled branched-chain amino acid(s), or a deuterium-labeled palmitic acid(s). The profiling probe(s) can include a molecule(s) that can be used by a living organism for biosynthesis. The molecule(s) can include (i) natural perdeuterated amino acids, (ii) partially deuterated amino acids, (iii) palmitic acid, (iv) oleic acid, (v) deuterated cholesterol, (vi) heavy water, (vii) deuterated glucose, (viii) deuterated acetate, (ix) alkyne bearing amino acids, (x) 1-homopropargylglycine, (xi) alkyne bearing fatty acids, (xii) 17-octadecynoic acid, (xiii) alkyne bearing nucleic acids, (xiv) 5-ethynyl-2′-deoxyuridine, (xv) 5-ethynyl uridine, (xvi) propargylcholine, (xvii) 3-O-propargyl-D-glucose, (xviii) Carbow orgenell dyes, (xix) Carbow-Mito, (xx) Carbow-Lyso, (xxi) Carbow-ER, (xxii) at least one drug, (xxiii) erlotinib, (xxiv) rhabduscin, (xxv) terbinafine, (xxvi) or carbonyl cyanide 4-(trifluoromethoxy)phenylhydrazone. In certain exemplary embodiments of the present disclosure, the profiling probe(s) can include, e.g., nanoparticles such as Rdots that can have Raman peak(s) that are separable from Raman peak(s) of the cells. In certain exemplary embodiments of the present disclosure, these nanoparticles can be biofunctionalized to target certain biomarkers or signaling pathways.

The exemplary profiling probe(s) can have a Raman peak(s) in a cell-silent spectral region. The cell-silent spectral region can be between 1800 cm⁻¹ and 2800 cm⁻¹. In certain exemplary embodiments of the present disclosure, a super-multiplexed Raman probe panel can include sharp and mutually resolvable Raman peaks to simultaneously quantify cell surface proteins, endocytosis activities, and metabolic dynamics of an individual live cell. When the exemplary super-multiplexed Raman probe panel is coupled to whole-cell spontaneous Raman micro-spectroscopy, the single-cell multiparameter measurement can enable powerful clustering, correlation, and network analysis with biological insights. The exemplary procedure can be used in, e.g., 14-plexed live-cell profiling and phenotyping under various drug perturbations.

According to certain exemplary embodiments of the present disclosure, a panel of super-multiplexed Raman probe can be provided that can target a wide range of key molecular and cellular markers. In certain exemplary embodiments of the present disclosure, by harnessing an ultra-bright Raman dots (Rdots) and with further functionalization and optimization, a panel of Rdots-conjugated antibodies and aptamers can be created. Additionally, ageneral exemplary procedure to detect cell surface proteins simultaneously in single live cells can be developed. Moreover, by leveraging the independent size and color tunability of Rdots and their good biocompatibility, profiling cellular endocytic pathways in a particle-size-dependent manner can be achieved. Furthermore, multiplexed metabolic activities can be profiled in parallel by including small vibrational probes (such as, e.g., alkyne and C-D bond) with minimal perturbation. When implemented with a tailored whole-cell spontaneous Raman micro-spectroscopy (Supplementary Video), this exemplary live-cell profiling platform can provide a strategy to acquire integrated information about cell surface protein abundance, endocytosis activities, and metabolic dynamics simultaneously at a relatively high speed (e.g., 3600 cells/hour). The application of this exemplary platform in 14-plexed live-cell profiling and phenotyping under various drug perturbations can be used, for example, in measuring multiparameter information, characterizing cell heterogeneity, revealing underlying correlation, and discovering mechanisms of drug actions. The exemplary profiling platform according to exemplary embodiments of the present disclosure can be compatible with, among others, live cell cytometry, of low instrument complexity and capable of highly multiplexed measurement in a robust manner. Additionally, the exemplary profiling platform according to the exemplary embodiments of the present disclosure can provide a valuable system for both basic single-cell biology and translation applications, such as, e.g., high-content cell sorting and drug discovery.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary schematic diagram of the exemplary automatic confocal micro-Raman system according to an exemplary embodiment of the present disclosure;

FIG. 1B is an exemplary diagram illustrating workflow of data acquisition and analysis according to an exemplary embodiment of the present disclosure;

FIG. 2A is an exemplary graph illustrating concentration profiles of d-PA and d-AA metabolites according to an exemplary embodiment of the present disclosure;

FIG. 2B is an exemplary dendrogram of 5 drugs with various mechanisms of action according to an exemplary embodiment of the present disclosure;

FIGS. 2C and 2D are exemplary graphs illustrating metabolic heterogeneity of lipid synthesis activity and protein synthesis activity, respectively, according to an exemplary embodiment of the present disclosure;

FIG. 3A is n exemplary graph illustrating concentration profiles of d-PA and d-AA metabolites for lipid and protein synthesis activity in cancer cell lines derived from different tissue according to an exemplary embodiment of the present disclosure;

FIGS. 3B and 3C are exemplary graphs illustrating metabolic heterogeneity of lipid synthesis activity and protein synthesis activity, respectively, in various cell lines according to an exemplary embodiment of the present disclosure;

FIG. 3D is an exemplary graph illustrating concentration profiles of d-PA metabolites and d-AA metabolites represent lipid and protein synthesis activity in breast subtypes according to an exemplary embodiment of the present disclosure;

FIGS. 3E and 3F are exemplary graphs illustrating metabolic heterogeneity of lipid synthesis activity and protein synthesis activity, respectively, in various cell lines according to an exemplary embodiment of the present disclosure;

FIGS. 4A and 4C are exemplary graphs illustrating certain LDA plots produced using a label-free method according to an exemplary embodiment of the present disclosure;

FIGS. 4B and 4D are exemplary graphs illustrating other LDA plots using the exemplary MAP procedure according to an exemplary embodiment of the present disclosure;

FIG. 5 is an exemplary diagram illustrating a Zig-Zag scanning mode according to an exemplary embodiment of the present disclosure;

FIG. 6A is an exemplary flow diagram of a method according to an exemplary embodiment of the present disclosure;

FIG. 6B is a diagram illustrating cell identification and localization according to an exemplary embodiment of the present disclosure;

FIGS. 7A-7C are exemplary graphs illustrating signal comparisons between previously-known Raman spectrometers and the exemplary whole-cell confocal micro-Raman spectrometer according to an exemplary embodiment of the present disclosure;

FIG. 8 is a set of spectra graphs according to an exemplary embodiment of the present disclosure;

FIGS. 9A and 9B are a set of exemplary spectral graphs illustrating spectra of deuterated branched-chain amino acids, deuterated non-branched-chain amino acids and deuterated palmitic acid metabolites in cells according to an exemplary embodiment of the present disclosure;

FIGS. 10A-10C are exemplary diagrams illustrating exemplary approaches to constructing datasets according to an exemplary embodiment of the present disclosure;

FIGS. 10D-10F are exemplary graphs illustrating learning curves of exemplary classifiers according to an exemplary embodiment of the present disclosure;

FIGS. 10G-10I are exemplary histograms of random permutation tests according to an exemplary embodiment of the present disclosure;

FIGS. 11A-11C are exemplary graphs illustrating model performance and discrimination for three human breast cancer cell lines according to an exemplary embodiment of the present disclosure;

FIGS. 12A-12F are exemplary graphs illustrating learning curves of various exemplary statistical models according to an exemplary embodiment of the present disclosure;

FIGS. 13A-13E are illustrations of exemplary preparation and physical properties of ten-colored Rdots according to an exemplary embodiment of the present disclosure;

FIGS. 14A-14H are illustrations and graphs of exemplary Rdots conjugated antibodies and aptamers for live-cell profiling of surface proteins of single cells according to an exemplary embodiment of the present disclosure;

FIG. 15 is an exemplary schematic diagram of the exemplary automatic confocal micro-Raman system according to another exemplary embodiment of the present disclosure which also illustrates an exemplary workflow for an automated whole-cell multiparameter measurement;

FIGS. 16A-16M are illustrations and graphs of exemplary endocytic profiling assays by multi-color Rdots according to an exemplary embodiment of the present disclosure;

FIGS. 17A-17F are illustrations and graphs of exemplary principle and exemplary implementation of multiparameter Raman profiling of individual living cells according to an exemplary embodiment of the present disclosure;

FIGS. 18A-18K shows exemplary live-cell multiparameter profiling which reveals cellular response characteristics for different drugs according to an exemplary embodiment of the present disclosure; and

FIG. 19 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION OF EXEMPLARY EMBODIMENTS

The exemplary system, method and computer-accessible medium, according to an exemplary embodiments of the present disclosure, can be used to evaluate the metabolism in single mammalian cells by coupling multiple vibrational metabolic probes with high-throughput single-cell Raman spectroscopy, termed as metabolic activity phenotyping (“MAP”). Instrument-wise, an automatic whole-cell confocal micro-Raman spectrometer was designed, which improves the throughput of single-cell metabolism measurement over commercial confocal scope by at least 100 times. Probe-wise, MAP method takes advantage of the newly developed vibrational probes of various metabolic pathways. These exemplary probes can be chemical analogs of their native counterparts and can be incorporated into all living cells without perturbing the biological activities. While they have been originally developed for vibrational imaging with subcellular resolution, (see, e.g., References 13-15), MAP integrates multiple of them with different colors into a high-throughput platform of single-whole-cell Raman spectroscopy, for the first time.

The exemplary whole-cell Raman spectroscopy system can be used to optically integrate the Raman signal over the entire cell without the need for subcellular resolution. This can provide high speed and throughput for single cell measurement. In contrast, standard commercial systems measure the signal over a very small space. In the exemplary system/apparatus, a large illumination spot (e.g., approximately 8 µm plus or minus 15%) was used to match the size of a whole mammalian cell (e.g., when disassociated it was approximately 10 um in diameter). In contrast, standard commercial systems generally utilize very tightly focused laser illumination (less than 1 µm). The illumination spot can also be between about 1 µm (plus or minus 15%) to about 5 um (plus or minus 15%), between about 5 µm (plus or minus 15%) to about 10 µm (plus or minus 15%), or between about 10 µm (plus or minus 15%) to about 100 µm (plus or minus 15%). Moreover, to accommodate the large beam size in the exemplary system/apparatus, a quasi-confocal detection geometry was achieved by using a relatively large pinhole (e.g., approximately 300 µm plus or minus 15%) to reject as much background as possible without losing the signal. In contrast, the standard pinhole size is less than 100 µm. The pinhole size can also be between about 50 µm (plus or minus 15%) to about 200 µ (plus or minus 15%), between about 200 µm (plus or minus 15%) to about 400 µm (plus or minus 15%), or between about 400 µm (plus or minus 15%) to about 1000 µm (plus or minus 15%).

Deuterium-labeled branched-chain amino acids (“d-AA”) and deuterium-labeled palmitic acid (“d-PA”) were selected to probe the metabolic activity of protein synthesis and lipid synthesis, whose metabolites take a large portion of cellular dry mass. By applying MAP, the measurement of metabolism at the single-cell level, and metabolic heterogeneity was evaluated under various drug treatments. The exemplary reproducible classification models can be achieved and they can be more robust than label-free Raman methods against the dominating batch-to-batch variation. Additionally, the successful classification of cancer cell types and heterogeneous breast cancer subtypes were demonstrated. The capability of MAP to explore metabolic profiles at single-cell level makes it be a valuable tool for basic single-cell studies as well as drug screening applications.

### Exemplary Results

### Exemplary Automatic Whole-Cell Confocal Micro-Raman Micro-Spectrometer Facilities High-Throughput Cell Metabolism Measurement

A sufficiently large data set can usually be beneficial for any robust statistical models, and the major obstacle for large Raman spectra data set can be the slow data acquisition procedure. For example, the traditional Raman spectrum can be collected in the confocal mode with a tightly focused laser beam to obtain high spatial resolution. Due to the weak Raman scattering cross-sections and the limit of laser power one can use before photodamage, the acquisition time of the individual Raman spectrum per pixel can usually be around dozens of seconds or sometimes even minutes. In this exemplary manner, scanning the confocal focal spot over the entire cell volume (e.g., in order to avoid intracellular heterogeneity) can take a rather long time.

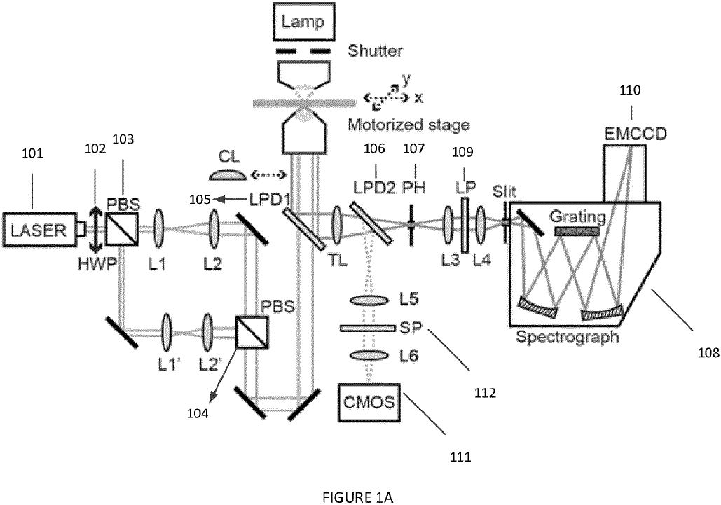

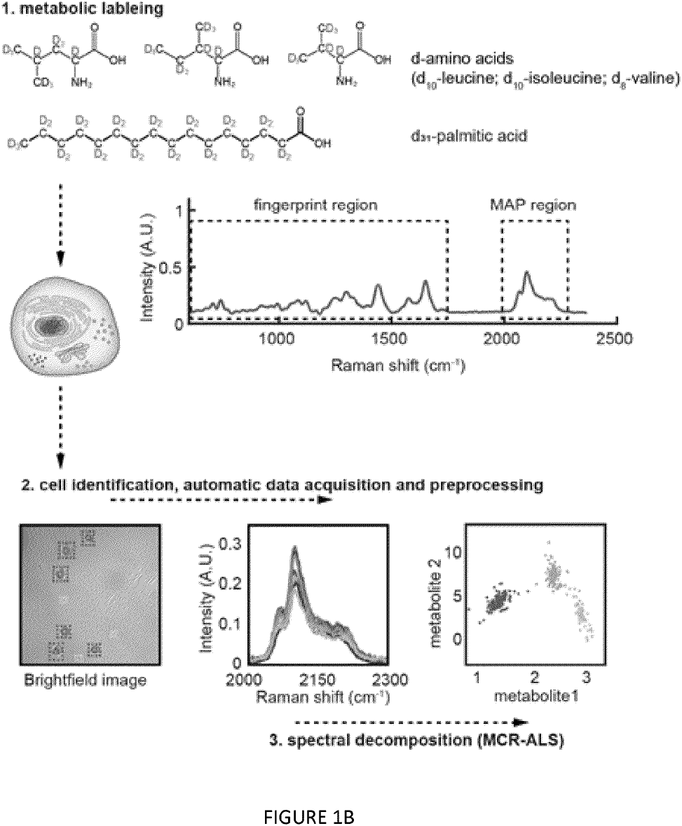

To tailor the exemplary procedure for high throughput cell phenotyping, an exemplary automatic whole-cell confocal micro-Raman spectrometer can optically integrate the signal over the entire cell without subcellular resolution. (See, e.g., FIG. 1A). In order to improve the signal to noise ratio and to reduce cell-to-cell variance, (see, e.g., References 16, 17), the illumination laser spot was expanded to approximately 8 µm so that the laser can roughly excite a whole mammalian cell. (See, e.g., Reference 18). In this exemplary manner, the optical signal can already be integrated over the entire cell, thereby improving the speed of acquisition. To accommodate the large beam size, a relatively large pinhole (e.g., 300 µm) was used for the quasi-confocal to reject as much background as possible without losing the signal. To reduce variance originated from intracellular inhomogeneity, a ‘zig-zag’ mode was employed using the motorized stage for those even larger cells. (See, e.g., FIG. 5). Additionally, a more powerful laser (e.g., 300 mW) was used to illuminate the entire cell (e.g., the power density can still be about 10-20 times smaller than that in conventional confocal Raman). A 600 lines/mm grating was used for its lesser angular dispersion and more photon accumulation on each pixel in the CCD as the result to additionally boost the acquisition time. (See, e.g., Reference 19).

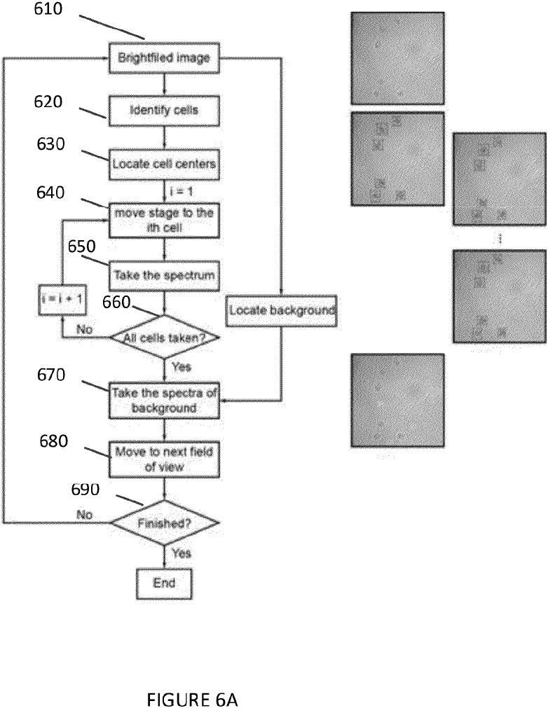

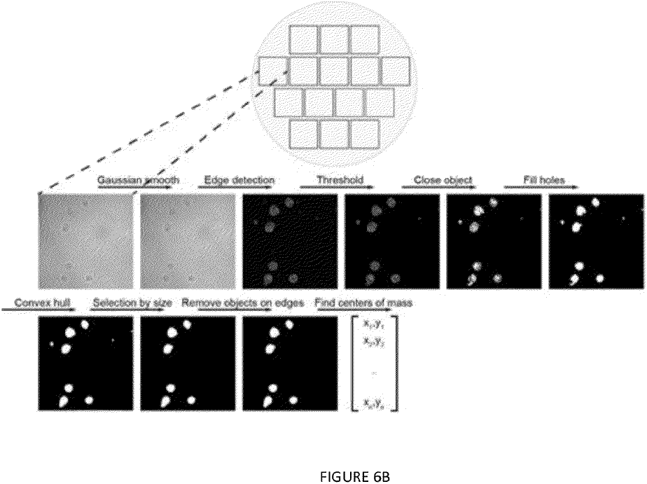

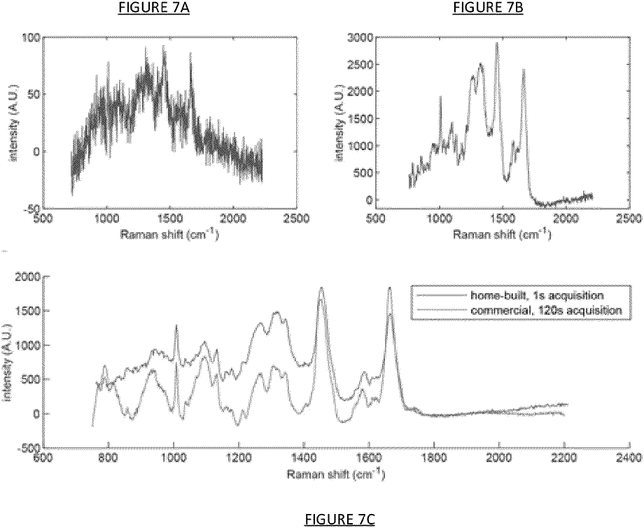

In addition, an exemplary procedure via software can be utilized with machine vision to automatically locate the cells and acquired data across the whole coverslip. (See, e.g., FIGS. 1B and 6). Compared to other approaches, such as, e.g., the Raman flow cytometer, this exemplary method can be inexpensive, and easily adopted in labs that may not have the expertise in microfluidics. As a result, the exemplary system, method and computer-accessible medium can can be used to acquire spectra from, e.g., at least 6000 cells (e.g., 0.5 s acquisition time) in an hour in a fully automatic manner.

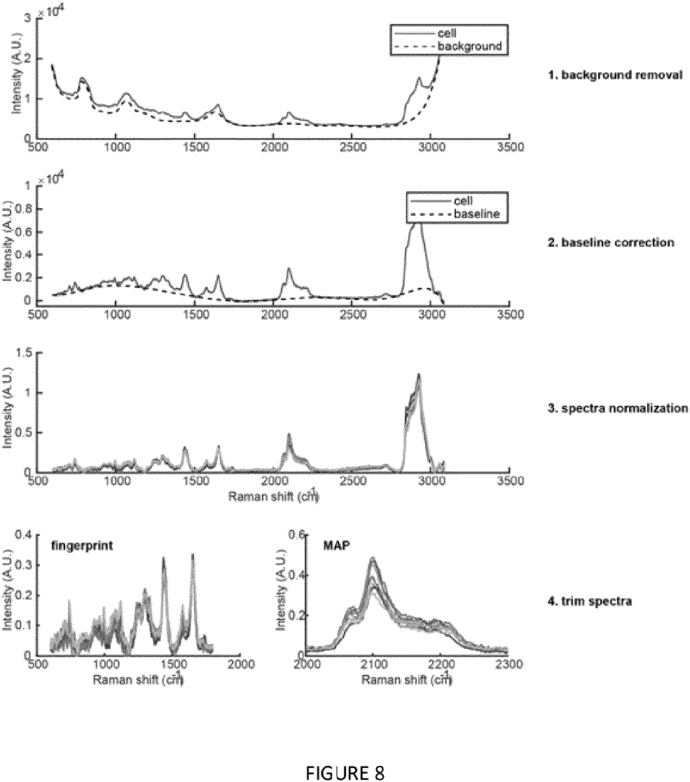

By combining the exemplary configurations and improvements described herein, the acquisition speed was improved to facilitate the high-throughput experiment, as well as an integration of the signal within a single cell in order to measure the metabolism at single-cell level reliably without the need of scanning either laterally or axially. Quantitatively, the acquisition speed was increased by at least 100 times compared to the commercial confocal system whose applicable power can be limited due to photodamage during slow acquisition. (See, e.g., FIGS. 7). This exemplary comparison can be made assuming only one spot in each cell can be sampled, however, since in conventional confocal Raman, the laser spot size can be around 300 nm, acquisition of multiple spots in one cell can be utilized to avoid intracellular variance, so the improvement of throughput could have been even larger.

FIGS. 7A-7C show exemplary graphs providing an exemplary signal comparison between commercially available micro-Raman spectrometer and home-built whole-cell confocal micro-Raman spectrometer. In particular, FIG. 7A illustrates an exemplary spectrum of a mammalian cell taken with Horiba XploRA PLUS micro-Raman spectrometer at 1 s acquisition time. FIG. 7B shows an exemplary spectrum of a mammalian cell taken with home-built whole-cell confocal micro-Raman spectrometer at 1 second acquisition time. The signal to noise ratio (SNR) can be, e.g., at least 100 times greater than the spectrum taken with commercially available Raman spectrometer. FIG. 7C illustrates exemplary normalized spectra taken with home-built Raman spectrometer and commercially available Raman spectrometer. To achieve same or similar level of SNR, it can usually take, e.g., 100 times longer exposure time with commercially available Raman spectrometer. Spectra with home-built Raman spectrometer were taken with 1 second acquisition time. Spectra with commercial Raman spectrometer were taken with 120 second acquisition time. Spectra of 10 cells were averaged.

### Exemplary MAP With Vibrational Metabolic Probes

The differentiation of cells in the multicellular organisms can be regulated by, e.g., gene expression patterns which ultimately leads to distinctive metabolism. By probing the metabolic flux with MAP, these distinctive features can be reflected and used for robust cell phenotyping. (See, e.g., References 20, 21). Indeed, it has been recently demonstrated that heavy water labeling in microorganisms facilitated the classification of microbes. (See, e.g., Reference 22). However, heavy water incorporation can be much slower for mammalian cells than in microorganisms. Hence different metabolic probes can be beneficial.

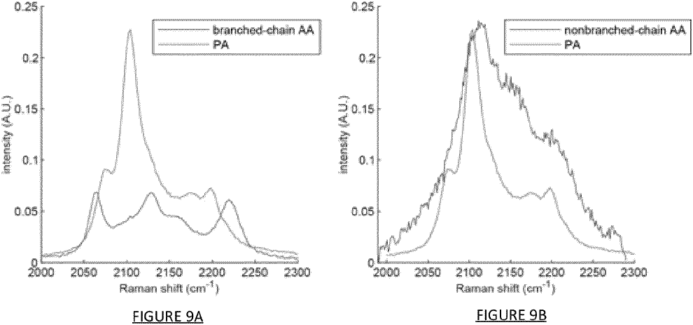

Deuterated branch-chained amino acids and deuterated palmitic acid were supplemented to the cell culture media for 24 hours in order to probe the flux of protein and lipid synthesis. There can be several considerations for choosing these two types of probes. First, protein and lipids can be the major components of cell dry mass and their metabolism can be tightly regulated. Second, Raman spectra of the metabolites of these two deuterated probes in cells can be shifted to the cell silent region (e.g., 2000 — 2300 cm-1), which facilitated the detection with high sensitivity in a background-free manner. (See, e.g., FIG. 8). Third, compared to non-branch-chained counterparts, branch-chained amino acids were chosen for their narrower Raman spectra in order to have better spectral differences from that of deuterated palmitic acid. (See, e.g., Reference 23; and FIGS. 9). With the high-throughput instrumentation efforts described above, Raman spectra of these two types of probes can be acquired as quickly as 0.5 s for the entire cell with a satisfactory signal to noise ratio. (See, e.g., FIG. 1B).

According to various exemplary embodiments of the present disclosure, a home-built automatic confocal micro-Raman system can be utilized. For example, in one exemplary and non-limiting implementation, a laser at 532 nm and a 600 lines/mm grating blazed at 500 nm was installed in the spectrometer. The back aperture was underfilled of the objective to expand the illumination volume (~8 um in diameter). The exemplary system was controlled by a generated software package in the Labview environment and is capable of automatic acquisition. An exemplary brightfield image was taken, and cells in the field of view were identified and located. Then, the exemplary computer-controlled motorized stage moved the slide so that illumination spot was parked on the cell, followed by Raman spectrum acquisition. This exemplary and non-limiting process was repeated for the next cell in the field of view until all the cells were taken.

FIGS. 9A and 9B show graphs of exemplary spectra of deuterated branched-chain amino acids, deuterated nonbranched-chain amino acids and deuterated palmitic acid metabolites in cells. For example, Deuterated branched-chain amino acids (see FIG. 9A) are spectrally more resolvable to deuterated palmitic acid compared to deuterated nonbranched-chain amino acids (see FIG. 9B). Most of the spectra of deuterated nonbranched-chain amino acids overlap with that of deuterated palmitic acid, making it more difficult to spectral decompose the two.

Turning FIG. 1B, this figure shows an exemplary illustration of an exemplary workflow of data acquisition and analysis according to an exemplary embodiment of the present disclosure. The exemplary workflow of FIG. 1B includes metabolic labeling (step 1), cell identification and automatic data acquisition and preprocessing (step 2), and spectral decomposition (step 3). For example, after cells were seeded, metabolic probes were added to the culture media. Then, the Raman spectra were acquired with the automatic confocal micro-Raman system. Cell silent region (2000 _{cm}⁻¹ ~ 2300 _{cm}⁻¹) was used for MAP analysis. Raman spectra were preprocessed, followed by MCR-ALS to decompose the spectra with pure components to obtain concentration profiles.

### Exemplary Direct Metabolic Activity Measurements in Single Cells Using MAP

Previously, the single-cell metabolic measurement has been challenging. Previously-used methods, such as mass spectrometry, can be difficult to reveal single-cell level heterogeneity. (See, e.g., Reference 24). Label-free Raman microscopy, in principle, reveals the integrated chemical composition in single cells and therefore offers information about single-cell metabolism. However, due to the complex nature of the Raman spectra, it can be difficult to assign each Raman peak in the fingerprint region to a specific chemical compound. (See, e.g., Reference 25). In addition, when dimension reduction approaches such as PCA can be applied, which can be a common practice for data analysis, the physical meanings of the dimension reduced data can be further lost. On the other hand, since the metabolic probes used have distinctive spectra, it can be straightforward to decompose the spectra in order to obtain the concentration information of each individual probe. Here the exemplary MCR-ALS method, (see, e.g., References 26-28), was used to decompose the composite spectra into the pure spectra of the two metabolic probes, and concentration profiles could thereby be obtained.

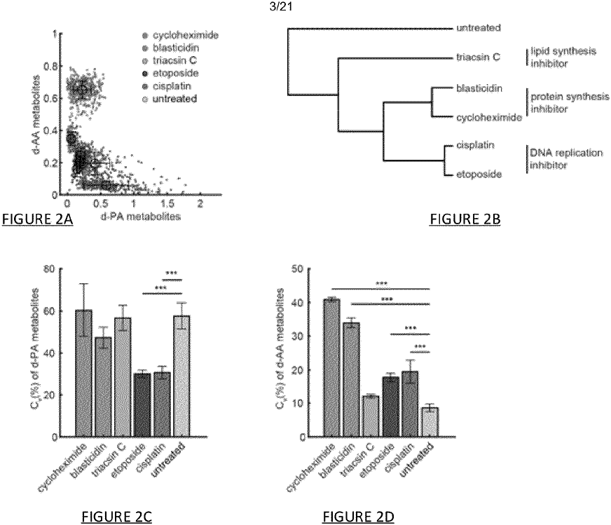

To validate the ability to reveal metabolic activity, the exemplary MAP was applied to cultured cells treated with drugs that inhibit known metabolism. Five drugs were used as the reference: cycloheximide and blasticidin, both inhibit protein translation; Triacsin C can be an inhibitor of long fatty acyl CoA synthetase; etoposide and cisplatin can be DNA replication inhibitors. MAP probes and individual drugs were added to the seeded A375 cells and allowed to incubate for 24 hours, after which the cells were fixed and Raman spectra were taken from each individual of the fixed single cells. At least 3 batches of each drug were prepared to ensure model robustness and reproducibility. MCR-ALS on the Raman spectra taken with MAP were applied, from which the concentration profiles of d-AA metabolites and d-PA metabolites in every single cell were calculated. (See, e.g., FIG. 2A).

Both cycloheximide and blasticidin show a significant decrease of d-AA metabolites compared to the untreated group (see, e.g., FIG. 2A, points 205 and 210), indicating that MAP indeed reveals protein synthesis activities. Similarly, cells treated with triacsin C show a significant loss of d-PA metabolites compared to the untreated cells (see, e.g., FIG. 2A, points 215)), indicating that MAP also reveals lipid synthesis activities. Combined, these interventions of metabolic inhibitor demonstrate the robustness and specificity of MAP in the high-throughput measurement of single-cell metabolism. To further validate the robustness of the exemplary MAP, hierarchical clustering analysis (“HCA”) was applied on the averaged spectra taken with MAP. (See, e.g., FIG. 2B). The results show that similar metabolic inhibitors were successfully clustered into the same nodes, indicating metabolic inhibitors of distinctive mechanisms of action can be distinguished reliably with MAP.

### Exemplary Mechanistic Insight Into Drug Response in Single Cells Using MAP

Cell-based assays are becoming increasingly important in drug discovery and drug studies for their better reflection of the complexity of the entire living organism than target-centric biochemical assays. (See, e.g., Reference 29). Due to the complex nature of cellular phenotypes, multiplexed measurements can be needed to comprehensively study the mechanism of the drugs in order to facilitate drug discovery. In addition, because cells can respond to the drug treatment differently because of their heterogeneous nature, which can give rise to the drug resistance, single-cell resolution can also be demanded to distinguish such heterogeneous response to better understand the efficacy. (See, e.g., Reference 4). In this regard, a procedure that can be to probe multiple metabolic states of single cells can be preferable.

As it has been shown that MAP can reliably probe the metabolic activity in single cells, a determination was made as to whether a metabolic inhibitor of a specific pathway can affect other pathways. Indeed, although triacsin C specifically inhibits long fatty acyl CoA synthetase, it shows a decrease in protein synthesis activity. This can be supportive of previous findings that suggest inhibition of lipid synthesis also leads to inhibition of cell proliferation and thereby lowers protein synthesis activity. (See, e.g., Reference 30). It was also observed that DNA replication inhibitors caused low protein synthesis activity. (See, e.g., FIG. 2A, points 220 and 225). This can be consistent with previous findings that inhibition of DNA replication reduces H1 histone mRNAs (see, e.g., Reference 31), which can be utilized for protein synthesis. These results reiterate the importance of multi-parameter measurement for a comprehensive view of drug response.

Since MAP measures the metabolic activity in single cells, its potential in quantitatively characterizing single-cell metabolic heterogeneity was determined. The coefficients of variation (“Cv”) of the single-cell d-PA metabolites and d-AA metabolites distribution were calculated from the metabolic inhibitor-treated cells. (See, e.g., Reference 32). The Cv value can be a normalized measure of the dispersion of the single-cell metabolic activity distribution and thus it can be used as a parameter to quantify the metabolic heterogeneity. Compared to the untreated control group, the treatment of cycloheximide and blasticidin did not alter the lipid synthesis activity distribution. Similarly, triacsin C did not change the metabolic heterogeneity in d-PA metabolites either, indicating a uniform efficacy of the drug. Etoposide and cisplatin, however, showed a significantly decreased lipid synthesis heterogeneity. (See, e.g., FIG. 2C). On the other hand, the two protein synthesis inhibitors, as well as the DNA replication inhibitors, showed a significantly elevated heterogeneity in protein synthesis metabolism, indicating a heterogeneous treatment effect. (See, e.g., FIG. 2D). Triacsin C, however, showed an unaltered Cv value compared to the control group. These results reaffirm the heterogeneous responses of cells to the drug treatment and demonstrate the potential of MAP to characterize single-cell metabolic heterogeneity.

FIGS. 2A-2D show illustration explaining exemplary metabolic activity and heterogeneity in cells treated with metabolic inhibitors measured by MAP. In particular, FIG. 2A illustrates exemplary concentration profiles of d-PA metabolites and d-AA metabolites represent lipid and protein synthesis activity in cells. Black circles indicate averaged value from single cells and error bars indicate standard deviation within the group. Pcycloheximide,lipid <0.0001; Pblasticidin,lipid <0.0001; Ptriacsin C,lipid <0.0001; Petoposide,lipid =0.0002; Pfisplatin,lipid =0.1. Pcycloheximide,protein <0.0001; Pblasticidin,protein <0.0001; Ptriacsin C,protein <0.0001; Petoposide,protein <0.0001; Pfisplatin,protein <0.0001. FIG. 2B shows an exemplary Dendrogram of 5 drugs with various mechanisms of action. Drugs having similar acting mechanisms are clustered into the same node while all drugs are separated from the untreated control group, indicating that MAP is sensitive to metabolic changes. FIGS. 2C and 2D illustrate bar graphs explaining exemplary metabolic heterogeneity of lipid synthesis activity and protein synthesis activity respectively in response to metabolic inhibitors. Cv values for each drug treated group were calculated, at least 3 batches were used to ensure statistical significance. Error bars represent standard deviation, *** indicates p value <0.005.

### Exemplary Metabolic Heterogeneity in Various Cancer Cell Lines by MAP

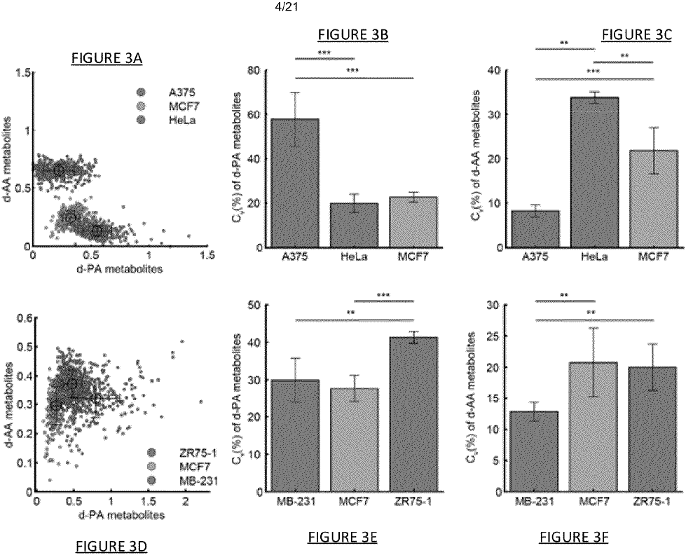

By validating that MAP can explore the metabolic activity and metabolic heterogeneity in drug-treated cells, the exemplary MAP was applied to measure the single-cell metabolism of commonly used cancer cell lines and breast cancer cell lines. Three different cancer cell lines were used: A375, HeLa and MCF7 that were respectively derived from skin cancer, cervical cancer, and breast cancer tissues. The lipid and protein synthesis activity of these three cell lines were measured with MAP. At least three batches of cells were used to ensure statistical significance. (See, e.g., FIG. 3A). The results suggest while HeLa cells can be more active in lipid metabolism, A375 cells have significantly higher activity in protein synthesis. The Cv of lipid synthesis and protein synthesis activity in these cell lines were calculated. A375 cells show significantly higher lipid synthesis heterogeneity compared to HeLa and MCF7 cell lines. (See, e.g., FIG. 3B). On the other hand, HeLa cells show the most heterogeneity in protein synthesis. (See, e.g., FIG. 3C).

Breast cancer can be one of the most common malignant cancer among females. The heterogeneity of breast cancer has been well recognized, and each subtype has different prognosis and treatment response due to their unique gene expression profiles and subsequent metabolism. Therefore, MAP was applied to breast cancer cell lines to investigate their metabolism profiles. Three breast cancer cell lines were chosen for their clinical significance: MCF7, a luminal A subtype; ZR-75-1, a luminal B subtype, and MDA-MB-231, a Claudin-low subtype. (See, e.g., Reference 33). Compared to the previously shown cells derived from different tissue, the breast cancer cell lines have similar lipid and protein metabolism levels. (See, e.g., FIG. 3D). Nevertheless, among the three cell lines, ZR-75-1 cells show the most abundant of d-PA metabolites, indicating an active lipid synthesis activity while MCF7 cells have the least lipid synthesis activity. MDA-MB-231 cells have the most protein synthesis activity among the three cell lines, and MCF and ZR-75-1 have similar level in protein synthesis. (See, e.g., FIG. 3D). These results suggest that the distinctive gene expression profiles in the breast cancer cell lines could lead to distinguishable metabolism.

The Cv values of lipid and protein synthesis in these cells were calculated to demonstrate their metabolic heterogeneity. (See, e.g., FIGS. 3E and 3F). It was found that ZR-75 cells have the largest Cv value among the three cell lines, indicating a more chaotic nature of its metabolic heterogeneity. On the other hand, MDA-MB-231 has the least Cv value in protein synthesis activity, suggesting a more uniformly distributed protein synthesis metabolism.

Overall, these exemplary results demonstrate the capability of MAP to explore the metabolic profiles in single cells. Because the procedure can be intrinsically based on single cells, measurements such as metabolic heterogeneity can be easily obtained. It was also found that it can be beneficial to quantify multiple parameters at the same in order to have a better understanding of the metabolic profile of a cell. In the exemplary measurement, for example, A375 cells have similar lipid synthesis activity but distinctive activity in protein synthesis compared to MCF7 cells. Likewise, the metabolic heterogeneity levels can be different between the two cell lines in both lipid and protein metabolism. Such information could have been overlooked if only one parameter was measured. Since the probes used, as well as other Raman metabolic probes, (see, e.g., Reference 13), have sharp and distinguishable peaks, it can be beneficial to use these probes for simultaneous multi-parameter measurement.

FIGS. 3A-3F show illustration explaining exemplary metabolic activity and heterogeneity in human cancer cell lines measured by MAP. For example, FIG. 3A illustrates exemplary concentration profiles of d-PA metabolites and d-AA metabolites represent lipid and protein synthesis activity in cancer cell lines derived from different tissue. FIGS. 3B and 3C show bar graphs of exemplary metabolic heterogeneity of lipid synthesis activity and protein synthesis activity respectively in various cell lines. Cᵥ values for cell line were calculated, at least 3 batches were used to ensure statistical significance. Error bars represent standard deviation, ** indicates p value <0.05, *** indicates p value <0.005. A375 cells show more chaotic lipid synthesis activity but less protein synthesis activity. FIG. 3D illustrates exemplary concentration profiles of d-PA metabolites and d-AA metabolites represent lipid and protein synthesis activity in breast subtypes. FIGS. 3E and 3F show bar graphs of exemplary metabolic heterogeneity of lipid synthesis activity and protein synthesis activity respectively in various cell lines. Cᵥ values for cell line were calculated, at least 3 batches were used to ensure statistical significance. Error bars represent standard deviation, ** indicates p value <0.05, *** indicates p value <0.005. ZR-75-1 cells show more heterogeneous lipid synthesis activity while MDA-MB-231 cells show least protein synthesis heterogeneity.

### Exemplary Stable Featured Among Batches and Reliable Discriminate Of Cancer Cells

Label-free methods, such as Raman-based vibrational microscopy, rely on physical and chemical properties to generate information about cell phenotypes. (See, e.g., Reference 12) and usually don’t demand a priori knowledge of biomarkers. For this reason, label-free Raman spectroscopy has been extensively investigated and successfully demonstrated in many diagnostic applications. (See, e.g., References 25, 34, 35). In the past decade, great efforts have been put into improving the hardware in order to increase the signal to noise ratio. (See, e.g., References 36-38), and into developing sophisticated statistical tools such as machine learning procedures to achieve better diagnostic results. (See, e.g., Reference 39). Yet challenges remain that hinder the prevalence of this powerful procedure in broader applications. Among those challenges, to achieve a robust and reproducible detection and classification classifier model can be the key.

Having shown that MAP can reveal the metabolic profiles in individual single-cells, it was determined that the method might combine the merits of the label-free Raman approaches and label-based approaches. Compared to pure physicochemical measurement, metabolic activities can reflect gene expression levels of many enzymes and proteins in the complicated metabolic network in an integrated manner, thereby revealing valuable information of cell phenotypes. In this sense, it was determined that the exemplary MAP can provide better sensitivity, reproducibility and interpretability than the conventional label-free Raman methods.

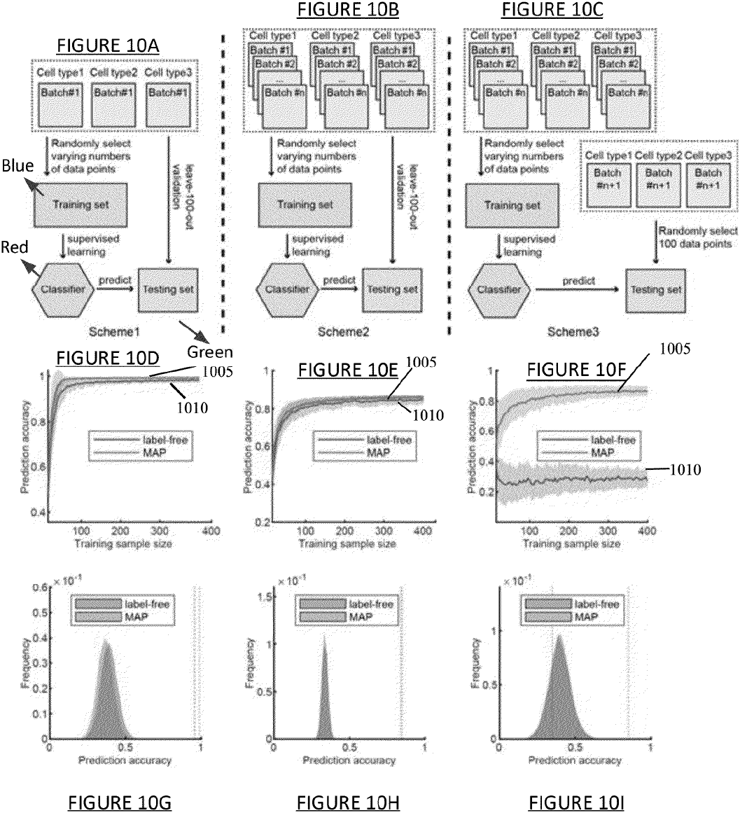

To investigate this, the model performance of three different data combination procedure (see, e.g., FIGS. 10A-10C) were compared with label-free Raman spectroscopy. Three different cancer cell lines: A375, HeLa and MCF7 were used to train a model that can differentiate these three cell types. A ‘batch’ of cells of a specific cell type were referred to a dish of cells that can be seeded on a particular day, and different ‘batches’ can mean that those cells were seeded on different days. Raman spectra of these batches of cells were put together to form the original data pool, from which an increasing number of samples were randomly withdrawn to construct the training set, and 100 samples that were not in the training set were randomly selected. Then a classifier model was trained with the training set and assessment was evaluated by using the trained classifier to predict the testing set. The process was repeated 100 times to ensure statistical significance.

FIGS. 10A-10I illustrate an exemplary classifier performance with three distinctive data combination schemes, with. FIGS. 10A-10C showing exemplary three approaches for constructing the datasets, while FIGS. 10D-10F illustrating exemplary learning curves of the classifiers corresponding to scheme 1, 2, and 3 respectively. Blue lines 1010 are learning curves with label-free method, and red lines 105 being learning curves with MAP. Shaded areas represent standard deviation. FIGS. 10G-10I show exemplary histograms of random permutation tests for scheme 1, 2 and 3 respectively. The model accuracy of the permuted ‘false dataset’ is much lower than the true dataset (blue dash lines, label-free true accuracy; red dash lines, MAP true accuracy), rejecting the null hypothesis and confirming the high prediction accuracy from the true dataset is real.

It was determined that the training and testing data can be from the same batches (see, e.g., FIGS. 10A-10C, scheme 1 and scheme 2), the trained models have high prediction accuracy (e.g., >85%). (See, e.g., FIGS. 10D and 10F). However, when the training and testing data can be from distinct batches (see, e.g., FIGS. 10A-10C, scheme 3), the label-free Raman fails to correctly classify (e.g., accuracy<40%) the three cell lines. (See, e.g., FIG. 10F). To further validate the observation, random permutation tests. (See, e.g., Reference 40) was carried out by firstly shuffling (e.g., permuting) the labels associated with each spectrum and then re-training the model using the shuffled dataset to make prediction in order to get the accuracy. Such a procedure was repeated 100,000 times to give the distribution of the accuracy under a null hypothesis. When the exemplary model from the real dataset can be reasonable, the prediction accuracy from the ‘false dataset’ can be expected to be low. Such exemplary permutation test also confirms that observation. (See, e.g., FIGS. 10G and 10H).

These exemplary observations indicate that batch-to-batch variation can be one of the major sources of variation for label-free methods. Data leakage happens when the training and testing datasets can be derived from the same batches, which leads to an overfitted model that can fail to predict the unseen samples. This can be in agreement with the findings from the Popp group (see, e.g., Reference 41), Smith group (see, e.g., Reference 42) and Fujita group (see, e.g., Reference 43). It was determined that this can be due to the fact that very often the spectral differences between cell types to be differentiated can be subtle, which can be subject to unpredictable changes if the system and specimen conditions cannot be precisely controlled.

The exemplary MAP classifier model performance was tested using the same data combination. (See, e.g., FIGS. 10A-10C). In schemes 1 and 2, MAP classifiers perform comparably to label-free classifiers. (See, e.g., FIGS. 10D and 10E, lines 1005). In scheme 3, however, while the learning curve shows that the label-free classifier model fails to classify the testing data, MAP classifier has a high accuracy (e.g., approximately 85%) predicting the unseen data. (See, e.g., FIG. 10F). Random permutation test also confirms the result (see, e.g., FIGS. 10G-10I): the model accuracy of the permuted ‘false dataset’ can be much lower than the true dataset, rejecting the null hypothesis and confirming the high prediction accuracy from the true dataset can be real. It’s worth noting that it utilizes a much larger sample size (e.g., approximately 300 cells) in scheme 3 to generate a stable classifier model, and the corresponding accuracy can be lower than that of procedure 1, which can be consistent with the determination that batch to batch variation can be the major variation.

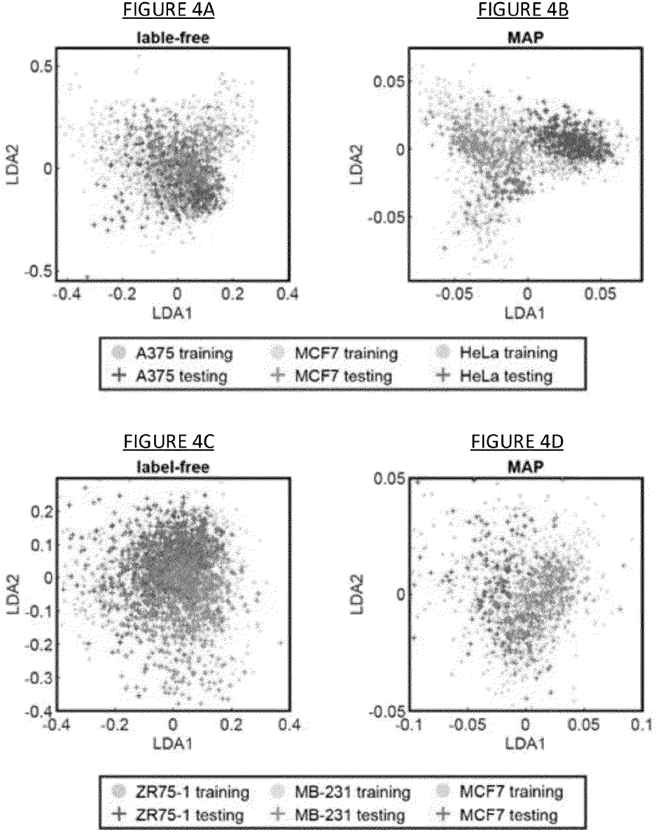

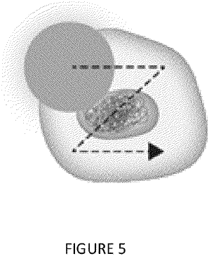

FIGS. 4A and 4B show exemplary acquired Raman spectra after a dimension reduction from the training datasets and the testing datasets with procedure 3 (ntrain=1539; ntest=479). Both exemplary methods successfully cluster the training samples into three distinct clusters as shown in the shaded dots. But for the testing data sets, the label-free method fails to correctly predict the identities of the data points shown in solid crosses, none of which can be overlapping to their corresponding training points properly. However, the testing data points with MAP illustrate overlapping with the training data and three clusters can be clearly separated correctly. It can be evident that MAP can distinguish the three cancer cell lines. These results demonstrate that MAP enables more stable and robust classification models that provide reproducible predictions over batches.

In particular, such FIGS. 4A-4C show graphs which provide an exemplary discrimination of human cancer cell types. Measured Raman spectra after dimension reduction with PCA and LDA are represented by the data points shown on the LDA1-LDA2 plane. Shaded dots represent training data, solid crosses represent testing data. FIGS. 4A and 4C illustrate exemplary LDA graphs with the label-free method, which failed to prediction the testing dataset. FIGS. 4B and 4D show exemplary LDA plots with MAP method, clearly showing three distinct clusters for both training and testing dataset. In example embodiments, only in MAP the testing data points have the same cluster as the training data, and therefore discrimination of cancer cell lines can be achieved.

### Exemplary Robust Discrimination Breast Cancer Cell Subtypes

The generality of exemplary MAP determined. Breast cancer can be one of the most common malignant cancer among females, and current diagnostic methods can be time-consuming and intrusive. (See, e.g., Reference 44). In addition, heterogeneity of breast cancer has been well recognized, and each subtype has different prognosis and treatment response. To address the screening challenge, vibrational spectroscopy has been extensively investigated for its non-intrusive nature. (See, e.g., References 44-46). Yet in previous studies using vibrational spectroscopy, the heterogeneous subtypes of breast cancer were overlooked. Therefore, three breast cancer cell lines were used for their clinical significance whose distinctive metabolism profiles have been demonstrated: MCF7, a luminal A subtype; ZR-75-1, a luminal B subtype, and MDA-MB-231, a Claudin-low subtype. (See, e.g., Reference 33). Spectra of these cells were acquired using both the label-free and MAP methods for at least 4 batches of each cell type in order to examine batch-to-batch variation.

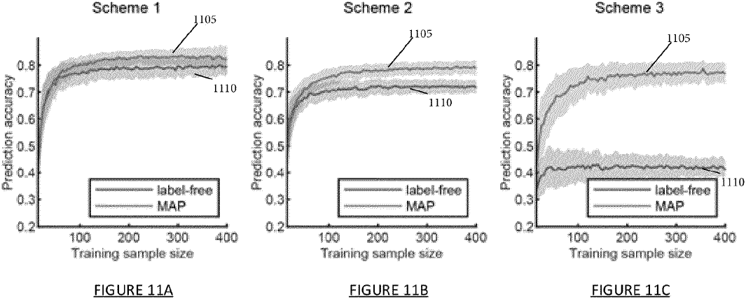

The learning curves were generated using the three different procedures. Akin to the exemplary previous results, good classifiers were acquired with procedure 1 and 2 with both label-free and MAP approaches. (See, e.g., FIGS. 11A-11C). Consistently, classifiers trained with procedure 1 have better accuracy than procedure 2, indicating the existence of batch-to-batch variation among these breast cancer cell lines. However, in procedure 3 which can be a more real-world application relevant, only with MAP can satisfactory classifiers be acquired. (See, e.g., FIGS. 11). These results once again confirm batch-to-batch variation and confirm that MAP preserves cell type features more robustly among batches of breast cancer cell lines.

Turning to FIGS. 4C and 4D, these figures show exemplary plots which demonstrate the differentiation of the three breast cancer cell lines with procedure 3. Evidently, both the label-free method and MAP can separate the training dataset reliably, but only with MAP, the testing dataset can be correctly discriminated.

FIGS. 11A-11C show exemplary graphs providing exemplary model performance and discrimination of three human breast cancer cell lines. In particular, as illustrated in FIGS. 11A-11C, exemplary learning curves of classifiers corresponding to scheme 1, 2, and 3, are provided, respectively. Blue lines 1110, learning curves with the label-free method; and red lines 1105, learning curves with MAP. Shaded areas indicate standard deviation.

### Exemplary Discussion With Exemplary Implementation

MAP can provide a direct quantitative measurement of metabolic activities at the single-cell level. With vibrational probes, it is possible to correlate the Raman spectra to the metabolism activities of individual cells so that the measurement of metabolism activities can be achieved. Furthermore, since the procedure can be at the single-cell level by nature, information such as cell heterogeneity can be readily available. As described herein, metabolic activities in each individual cell were measured, and the heterogeneity among each cell line or among each drug treatment was revealed. Coupled with the specifically designed automatic whole-cell confocal micro-Raman spectrometer, such measurement and analysis can be carried out in a high-throughput manner (e.g., with at least 100 times improvement over the commercial confocal system).

There may exist a deep reason underlying the good performance of MAP with vibrational metabolic probes. This exemplary procedure probes metabolic activities that can be regulated by transcriptomics, which can be one of the fundamental characters of organisms. In addition, because the probes may only be added for a short period of time (e.g., less than or similar to the doubling time of the cells), MAP can detect the passage of these metabolites through reactions system over time — the metabolic flux, which can be one of the most effective methods and has been used in a diversity of fields. (See, e.g., Reference 47). Moreover, protein and lipid metabolism can be among the most complexes processes in living organisms and can be tightly regulated by a large variety of signaling pathways during different cell stages. (See, e.g., References 48, 49). Therefore, by probing these metabolisms, integrated information of many metabolic enzymes in the network can be acquired about the cells.

Two categories of deuterium-labeled metabolic probes were used. However, the possible metabolic probes may not be limited to these two. Through the course of the past decade, numerous vibrational tags that serve as metabolic probes (e.g., propargylcholine and glucose) have been developed and demonstrated individually (see, e.g., Reference 13), many of which have distinctive spectral features that facilitated spectral decomposition. It was anticipated that a more comprehensive metabolism profile can be understood if more of these probes can be used simultaneously since richer information about the metabolic activities of diverse biochemical pathways can be revealed. Moreover, technological advances in instrumentation can provide faster acquisition and higher throughput. For example, coherent Raman processes, such as stimulated Raman scattering coupled with flow-cytometry, has been demonstrated with superior acquisition speed recently. (See, e.g., References 37, 50, 51). When using together with MAP according to exemplary embodiments of the present disclosure, even higher throughput can be achieved for more accurate screening. Together, the exemplary MAP can be a powerful tool for basic single-cell biological studies.

To further validate the exemplary model according to exemplary embodiments of the present disclosure, a random permutation test. (See, e.g., Reference 62) was carried out by firstly shuffling (e.g., permuting) the labels associated with each spectrum and then re-training the model using the shuffled dataset to make prediction in order to get the accuracy. Such exemplary procedure was repeated 100,000 times to give the distribution of the accuracy under a null hypothesis. When the exemplary model from the real dataset can be reasonable, the prediction accuracy from the ‘false dataset’ can be expected to be low.

In procedure 1, e.g., only one batch of each cell type was used to form the original data pool, and the classifier was trained using randomly selected samples from the data pool. (See, e.g., FIG. 10A). The learning curve shows that as the training sample size increases the model prediction accuracy also increases until it reaches the plateau when the sample size can be large enough (e.g., approximately 70 cells). (See, e.g., FIG. 10D, line 1010). At this point, the trained model has a high accuracy predicting the testing data (e.g., approximately 98%). The permutation test shows that if the labels of the samples can be randomly permuted, the exemplary model can fail to predict, ruling out the possibility of false positive. (See, e.g., Reference 62; and FIG. 10G). In procedure 2, at least 3 batches spanning months of each cell type were used to form the original data pool, from which the training and testing sets were randomly withdrawn. (See, e.g., FIG. 10B). Similar to procedure 1, the model performance increases as the sample size grows. (See, e.g., FIG. 10E, line 1010). However, the prediction accuracy reaches the plateau at approximately 150 cells, much later than it does in procedure1. Noticeable as well can be that the best accuracy can still be lower than that in procedure 1, hinting that more variation exists among batches. Likewise, the permutation test validates the model to rule out false positive. (See, e.g., FIG. 10H). In procedure 3, the training set was randomly drawn from a data pool consisting of at least 3 batches of cells. The testing set, however, was selected randomly from a different data pool consisting of batches of cells that were not in the training set. This procedure mimicked a real-life application scenario where a classification model was first trained with existing data and then used to identify the unseen samples. The learning curve for this procedure shows, however, as the training size increases, the model does not improve, and never reaches a plateau of high accuracy even when the training set consists of thousands of samples. (See, e.g., FIG. 10F, line 1010). Cross-validation using the training set alone, on the other hand, shows a high model accuracy (e.g., approximately 90%).

### Exemplary Cell Culture

HeLa, MCF7, A375, MDA-MB-231 were cultured in Dulbecco’s Modified Eagle’s medium (e.g., DMEM: 4.5 g/L glucose; 11965, Gibco) supplemented with 10% Fetal bovine serum (e.g., FBS; 16000, Gibco) and 1% antibiotics (e.g., Penicillin-Streptomycin-Glutamine; 10378016, Gibco). ZR-75-1 cells were cultured in RPMI 1640 medium (e.g., 11875, Gibco) supplemented with 10% Fetal bovine serum (e.g., FBS; 16000, Gibco) and 1% antibiotics (e.g., Penicillin-Streptomycin-Glutamine; 10378016, Gibco). Cells were maintained in 5% CO2 at 37° C. All cells were passaged 2-3 times a week and were never allowed to grow more than 90% confluency. Cells were tested mycoplasma negative.

HeLa cells were obtained from ATCC, MDA-MB-231 and A375 cells. MCF7 and ZR-75-1 cells were also obtained.

### Exemplary Cell Sample Preparation

Freshly disassociated cells were used to fill the as much the illumination volume as possible in order to maximize Raman signal to noise ratio since disassociated cells were usually round and thicker.

For label-free samples, cells were first seeded in a 24-well plate at 50% confluency with cell culture media. After overnight culture, cells were disassociated by TrypLE (e.g., 12604, Gibco). Then cells were allowed to settle onto 18 mm round quartz coverslips (e.g., 103302-258, Electron Microscopy Sciences) that were pre-coated with poly-lysine for 10 min. Cells were subsequently washed twice with PBS (e.g., 14040, Gibco) and fixed with 4% freshly diluted paraformaldehyde (e.g., PFA; 15713S, Electron Microscopy Sciences), followed by 3 times wash with PBS. Double-sided adhesive imaging spacers (e.g., GBL654002, Sigma) were used to make ‘sandwich’ samples with glass slides.

For MAP samples, cells were first seeded in a 24-well plate at 50% confluency with cell culture media and allowed to adhere overnight. Then the culture media was replaced with MAP culture media (see Table 1 below) and cells were further cultured in MAP media for 24 hours. For the drug tests, individual drugs (e.g., concentrations in Table 2 shown below) were added to the MAP media and cells were subject to the drug treatment for 24 hours. After the 24-hour culture, cell samples were prepared the same way as described above.

TABLE 1

MAP media formulation

| Components | Concentration (mg/L) |
|---|---|
| Amino Acids | |
| Glycine | 30 |
| L-Arginine hydrochloride | 84 |
| L-Cystine 2HC1 | 63 |
| L-Glutamine | 584 |
| L-Histidine hydrochloride-H2O | 42 |
| L-Lysine hydrochloride | 146 |
| L-Methionine | 30 |
| L-Phenylalanine | 66 |
| L-Serine | 42 |
| L-Threonine | 95 |
| L-Tryptophan | 16 |
| L-Tyrosine disodium salt dihydrate | 104 |
| Vitamins | |
| Choline chloride | 4 |
| D-Calcium pantothenate | 4 |
| Folic Acid | 4 |
| Niacinamide | 4 |
| Pyridoxine hydrochloride | 4 |
| Riboflavin | 0.4 |
| Thiamine hydrochloride | 4 |
| i-Inositol | 7.2 |
| Inorganic Salts | |
| Calcium Chloride (CaCl2) (anhyd.) | 200 |
| Ferric Nitrate (Fe(NO3)3″9H2O) | 0.1 |
| Magnesium Sulfate (MgSO4) (anhyd.) | 97.67 |
| Potassium Chloride (KCl) | 400 |
| Sodium Bicarbonate (NaHCO3) | 3700 |
| Sodium Chloride (NaCl) | 6400 |
| Sodium Phosphate monobasic (NaH2PO4—H2O | 125 |
| Other Components | |
| D-Glucose (Dextrose) | 4500 |
| Fetal Bovine Serum, charcoal stripped | 10% |
| Deuterated Vibrational probes | |
| L-Isoleucine-d10 | 105 |
| L-Leucine-d10 | 105 |
| L-Valine-d8 | 94 |
| palmitic acid-d31 | 50 mM |

TABLE 2

Drug final concentrations utilized

| | |
|---|---|
| cis-platin | 125 uM |
| blasticidin | 10 ug/ml |
| cycloheximide | 100 uM |
| triacsin C | 5 uM |
| etoposide | 50 uM |

### Exemplary Automatic Confocal Micro-Raman Spectrometer

Referring back to FIG. 1A which illustrates a schematic diagram of the exemplary confocal Raman microscope according to an exemplary embodiment of the present disclosure, a 532 nm laser 101 (e.g., Samba 532 nm, 400 mW, Cobolt Inc.) was used as the light source. The laser beam was first collimated and expanded by the telescope lenses (e.g., L1, L2/L1′, L2′, Thorlabs). A half-wave plate 102 (e.g., HWP, Thorlabs) and polarizing beamsplitters 103 and 104 (e.g., PBS, Thorlabs) were used to switch between the two beam expansion ratios (e.g., L1, L2 or L1′, L2′) to achieve two different illumination spot sizes. An exemplary objective was underfilled and the illumination spot had a diameter of approximately 8 um as the result. The expanded beam was then directed to the inverted microscope (e.g., IX71, Olympus) installed with a dichroic beamsplitter 105 or 106 (e.g., LPD1, LPD2-532RU-25, Semrock). The emitted Raman signal first passed a pinhole 107 (e.g., PH, 300 um, Thorlabs) for background suppression and was relayed by two lenses (e.g., L3, L4, Thorlabs) before being projected to the spectrometer 108 (e.g., Kymera 328i with 600 lines/mm grating blazed at 500 nm, Andor). A long-pass filter 109 (e.g., LP, LP03-532RU-25, Semrock) was installed between the relay lenses to block laser light from Rayleigh scattering. Raman signal was then collected by an EMCCD 110 (e.g., Newton970, Andor). For brightfield imaging, a long-pass dichroic (e.g., LPD2, FF511-Di01, Semrock) was installed in front of the pinhole and a set of relay lenses (e.g., L5, L6) were used to project the brightfield images to the CMOS 111 camera (e.g., DCC1645C, Thorlabs). A short-pass filter 112 (“SP”) was installed to suppress ghost images.

FIG. 6A shows an exemplary flow diagram of a method according to an exemplary embodiment of the present disclosure. In order to automatically acquire spectra from cells, as shown in FIG. 6A, a brightfield image was taken (610) and the cells in the images were identified (620) and their locations were subsequently calculated (630). (See, e.g., FIGS. 5, 6A, and 6B). A motorized stage (e.g., HLD117, Prior) moved the sample slide so that the center of a cell aligned with the illumination spot (640), after which the spectrometer was triggered to acquire the spectrum (650). The exemplary procedure cycled until all cells within a field of view were taken (660). Background spectra were taken for the field of view for spectral background removal (670). The stage then moved to the next field of view (680) and cycle through the procedures until the whole desired area was covered (690). (See, e.g., FIGS. 5 and 6). The process above was automated by a home-built software coded with Labview (e.g., National Instruments).

FIG. 6B shows a diagram illustrating multiple images providing cell identification and localization according to an exemplary embodiment of the present disclosure. In such exemplary embodiment, machine vision can be used to find the coordinates of the location of cell.

### Exemplary Preprocessing Procedure(s)

Exemplary preprocessing protocols were used to preprocess the raw spectra. (See, e.g., References 64 and 65). Prior to the preprocessing procedures, the spectra were calibrated by 1:1 mixture of acetonitrile and toluene taken on the same day. Thereafter, first, the quartz and water background were removed via an automatic procedure. (See, e.g., Reference 66). Second, an iterative procedure was used for baseline correction. (See, e.g., Reference 67). Third, the spectra were normalized to their amide peak. And lastly, the spectra were trimmed for subsequent analysis. The silent region (e.g., 2000 — 2300 _{cm}⁻¹) was used for MAP analysis and fingerprint region (e.g., 600 — 1800 _{cm}⁻¹) for label-free analysis. (See, e.g., FIG. 8).

FIG. 8 is a set of spectra graphs according to an exemplary embodiment of the present disclosure.

### Exemplary Machine Learning Model

In brief, once the training datasets were obtained, principal component analysis (“PCA”) was applied, followed by linear discrimination analysis. The k-nearest neighbor (“k-NN”) classifiers were then built on the dimension reduced data. To obtain the classifiers prediction accuracy, PCA and LDA were first applied to the testing dataset, then the trained classifiers were used to predict the labels of the test data, which were then compared to their known true labels to calculate the accuracy.

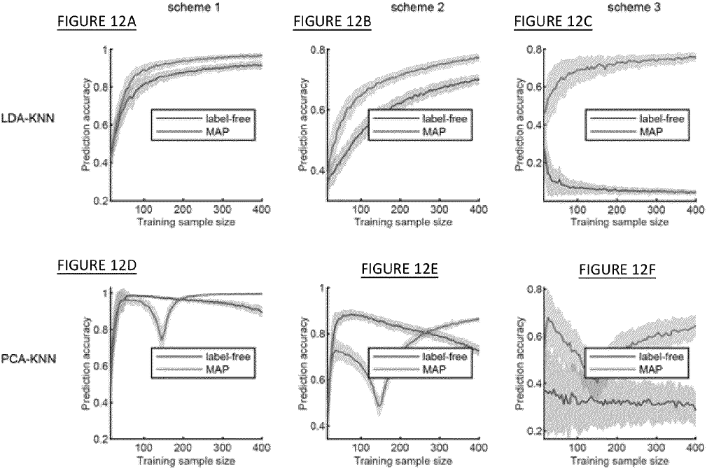

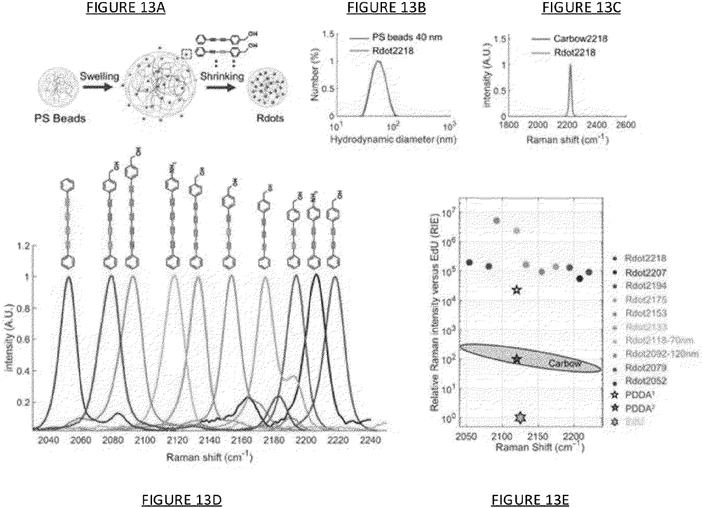

PCA has previously widely used to reduce noise and to improve statistical model robustness. By applying PCA to the training data, the training spectra were decomposed into multiple representations (e.g., scores and loading vectors) that capture as much of the information as possible. The number of components n was chosen in such a way that the accumulative variance explained was greater than 99%. Then the top n PCA scores were used as the input of LDA, which can be a supervised procedure that maximizes the distance between group means while minimizing the variance within a group. LDA scores and projection vectors can be obtained by the procedure, and LDA scores were further used for constructing k-NN clustering classifiers. To predict the test dataset, mean-centered spectra in the testing sets were first projected to PCA loading vectors obtained earlier in the training stage, and the PCA scores were further projected to training LDA projection vectors to obtain LDA scores for the testing sets. Then the dimension reduced data were input to the learned k-NN model to make the prediction. The model accuracies were then calculated by calculating the proportion of correctly predicted number of cells to the total number of cells. (See, e.g., FIGS. 12A-12F)

FIGS. 12A-12F show a set of exemplary learning curves of various statistical models according to various exemplary embodiments of the present disclosure. As shown in the FIGS. 12A-12C, classifiers were built with consecutive application of LDA and KNN without PCA. In FIGS. 12D-12F, classifiers were built with consecutive application of PCA and KNN without LDA.

### Exemplary Design and Characterization of Expanded Panel of Rdots Probes

Despite the high potential for multiplexing, the biological application of Raman spectroscopy may be limited by its small Raman cross-section, which is typically 10⁸ ~10¹⁴ times smaller than that of fluorescent dyes. (See, e.g., Reference A31). To amplify the signal, a procedure to prepare ultra-bright Raman dots (Rdots) by non-covalently doping polymer nanoparticles with Raman active dyes can be applied. (See, e.g., Reference A32). When coupled with Stimulated Raman Scattering (SRS) microscopy, these Rdots can show, among others, unprecedented imaging performance in immunostaining of intracellular targets. Exemplary ultra-bright Rdots can be harnessed for developing a robust and cost-effective platform for live-cell profiling. For the current exemplary application of live cell profiling, Rdots were tailored in, e.g., four aspects. First, unlike imaging intracellular targets where an extremely compact size of Rdots is necessary, the nanoparticle size requirement for profiling living cell surface proteins can be flexible. Therefore, the Rdots that were 40-120 nm in diameter were prepared to further increase their Raman brightness and facilitate the demonstration of endocytosis in a size-dependent manner. Second, the previously reported 6-color Rdots palette were expanded to 10 colors for the need of profiling cell surface proteins and endocytosis. Third, the surface functionalization has also been optimized for the new applications. Fourth, the current exemplary application of live cell profiling moved from immunostaining of fixed cells to profiling living cells.

FIGS. 13A-13E show illustrations of exemplary preparation and physical properties of ten-colored Rdots. For example, FIG. 13A provides an exemplary schematic illustration of a Rdots preparation via a swelling-shrinking strategy. FIG. 13B shows a exemplary dynamic light scattering (DLS) size characterization of Rdot2218 before and after dye doped. FIG. 13C shows exemplary Raman spectra of Rdot2218 in water and free Carbow2218 dye in DMSO. FIG. 13D shows exemplary normalized spontaneous Raman spectra of ten-colored Rdots, with corresponding doped dye structure listed above. FIG. 13E shows exemplary RIE values and Raman peak positions of ten-colored Rdots. Most Rdots exhibit tremendously high RIE values over 105, at least 5 times larger than PDDA36. Due to the volume effect, Rdots with larger sizes (70 nm and 120 nm) have an RIE value over 106. RIE values were measured with a spontaneous Raman spectrometer with 532 nm excitation. PDDA1 is the RIE value detected under the resonance Raman effect. PDDA2 is the RIE value detected without resonance enhancement.

The multi-colored Rdots were prepared via a swelling-shrinking strategy. (See, e.g., Reference A33). After the addition of swelling agent THF, adequate Carbow dyes can be incorporated into the swollen polystyrene (PS) beads. Subsequent shrinking and trapping of dyes were achieved by suspension in a large amount of water (FIG. 13A). After doping with Carbow dyes, the Rdots were dispersed evenly without obvious size expansion (see FIG. 13B). The Raman peaks of Rdots coincided well with free Carbow dyes in DMSO (see FIG. 13C), indicating the maintenance of spectral characteristics after being doped into PS beads. The zeta potential measurements of pure beads and Rdots proved that the surface carboxyl groups were mostly retained after the preparation process, facilitating appropriate colloidal stability and compatibility to subsequent bioconjugation. As shown in FIG. 13D, ten Carbow dyes were doped (see, e.g., Reference A25) with distinct Raman frequencies into 40 nm, 70 nm, and 120 nm PS beads to prepare ten-colored Rdots, with each Rdot being spectrally resolvable in the Raman-silent window (1800-2600 cm⁻¹). (See, e.g., Reference A34). The brightness of Rdots was evaluated through its relative Raman intensity versus EdU (RIE). (See, e.g., Reference A35). By virtue of the large Raman cross-sections and high local concentration of Carbow dyes enriched inside, most Rdots can exhibit tremendously high RIE values over 10⁵ (FIG. 13E), orders of magnitudes higher than the organic Raman probes developed recently. (See, e.g., References A25, A36). As a result, a nanoparticle-based Raman color palette was achieved in Figure 1d with highly multiplexing capability and ultra-brightness.

### Live-Cell Profiling of Surface Proteins of Single Cells by Rdots Conjugates

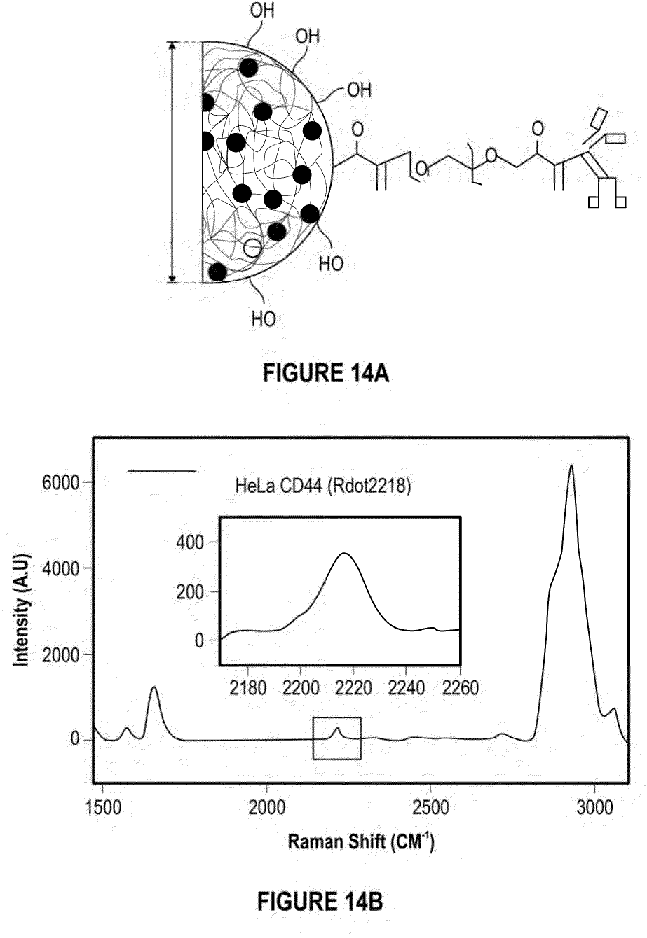

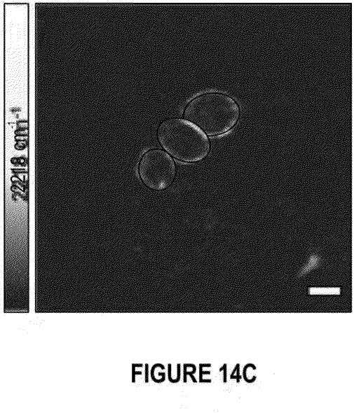

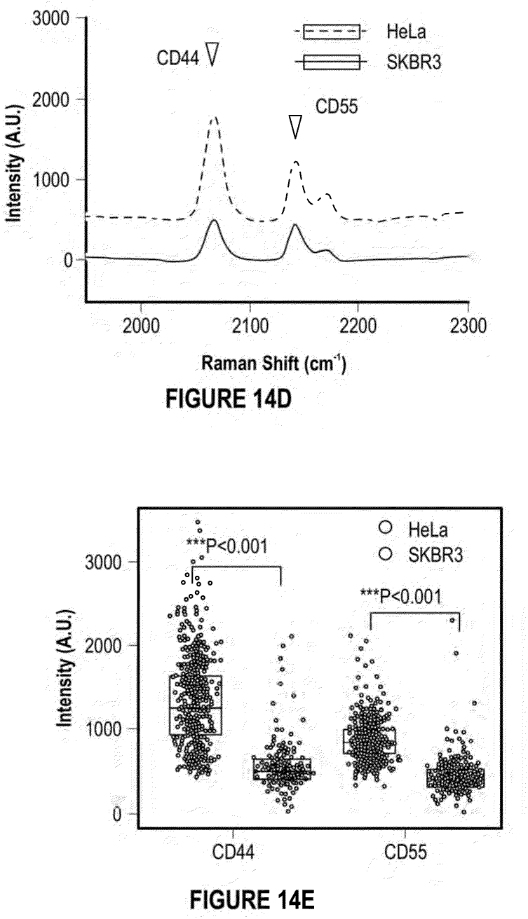

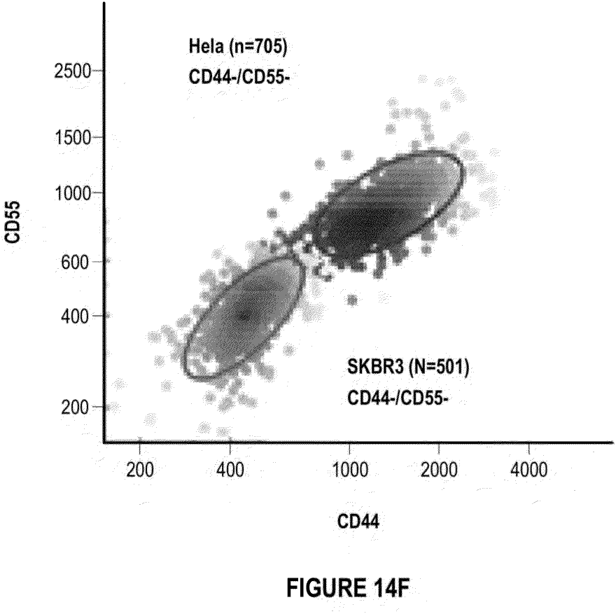

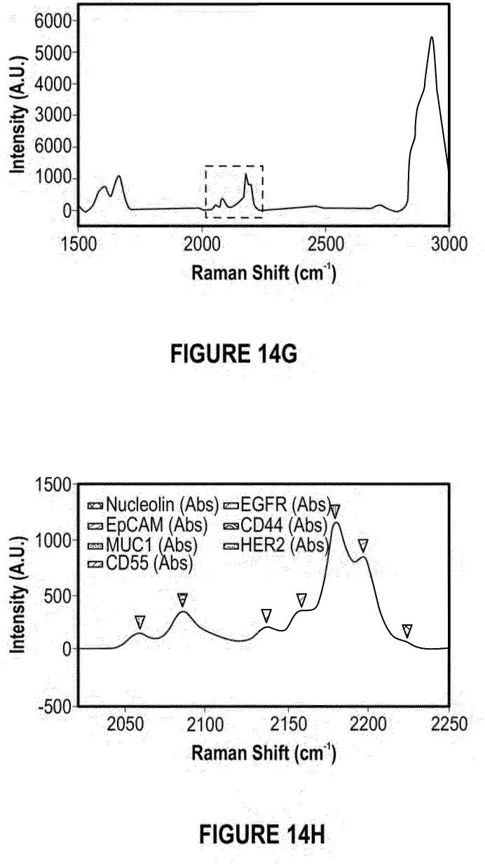

FIGS. 14A-14H show illustrations of exemplary Rdots conjugated antibodies and aptamers for live-cell profiling of surface proteins of single cells. For example, FIG. 14A shows an exemplary structure of Rdots conjugates. Carboxylated Rdots were first attached with amine-PEG-alcohol and amine-PEG-acid at a molar ratio of 9:1. The PEGylated Rdots were then conjugated with targeting molecules (antibody or aptamer) for recognition. FIG. 14B shows exemplary spontaneous Raman spectra of Rdot2218-CD44 stained HeLa cell, averaged over 100 individual cells. Excitation: 532 nm (400 mW), acquisition time: 1 s. FIG. 14C shows an exemplary SRS image of Rdot2218 positively staining of CD44 on HeLa cell surface, scale bar: 10 µm. FIG. 14D shows exemplary spontaneous Raman spectra of HeLa and SKBR3 cells acquired after dual-color staining. The shaded area indicates the standard deviation of Raman spectra from multiple cells (N_{HeLa}=705; N_{SKBR3}=501). Excitation: 532 nm (400 mW), acquisition time: 2 s. FIG. 14E shows an exemplary box plot illustrating the Raman intensity of CD44-targeted Rdot2079 and CD55-targeted Rdot2153 on HeLa and SKBR3 cell surface with *** denoting a significant difference between two groups (P<0.001). FIG. 14F shows exemplary two-dimensional scatter plot of individual cells, based on the quantitative readouts of two protein channels. FIGS. 14G and 14H show exemplary spontaneous Raman spectra of SKBR3 cells acquired after stained with seven Rdots conjugates, averaged over 600 individual cells (g), and zoom-in view (h). Excitation: 532 nm (400 mW), acquisition time: 1 s.

To profile living cell surface proteins, prepared Rdots were functionalized with antibodies or aptamers. A non-toxic and hydrophilic polymer polyethylene glycol (PEG) was employed to reduce potential nonspecific hydrophobic interaction and electrical adsorption of Rdots onto cell membrane. (See, e.g., Reference A37). As shown in FIG. 14A, the surface of 40 nm carboxylated Rdots were functionalized with long 5 kDa amine-PEG-acid and backfilled with short 1 kDa amine-PEG-alcohol to create a cage-like shell with EDC/NHS coupling chemistry. The surface coverage of the PEG layer minimizes the non-specific binding and supports a flexible spacer arm for an oriented bioconjugation to facilitate targeting efficiency. For protein recognition, the surface amine-PEG-acid was then covalently conjugated with targeting antibodies or aptamer with EDC/NHS chemistry.

Rdots have been imaged by narrowband SRS microscope in which only one Raman channel can be imaged at a time. (See, e.g., Reference A32). In this disclosure, single-cell Raman spectra of all vibrational modes were acquired in a cost-effective manner by employing whole-cell spontaneous Raman spectroscopy. (See, e.g., Reference A38). The illumination laser spot was intentionally expanded to 8~10 µm to ensure the excitation of the whole mammalian cell. By doing so, the Raman signal over the entire cell volume can be optically integrated as an individual spectrum with rapid acquisition of multiplexed probes, suitable for high-throughput single-cell applications. To test the feasibility of the detection, PEGylated Rdots were firstly conjugated with a primary antibody against CD44, a cell surface adhesion molecule that frequently overexpressed on cancer cells. (See, e.g., Reference A39). Rdot2218-CD44 stained HeLa cells exhibit a single, narrow and strong Raman peak in the cell-silent window under this setup (see FIG. 14B), thanks to the ultra-brightness of Rdots and optical integration of all Rdots labeled to single cells. FIG. 14C illustrates a corresponding SRS image at 2218 cm⁻¹, and the membrane-bound SRS signal confirms the subcellular localization of CD44 receptors and validates that Rdots recognition was specific enough for the detection of membrane proteins.

As a proof of concept of two-channel cell profiling, HeLa and SKBR3 cell lines stained with both Rdot2153-CD55 and Rdot2079-CD44 were assessed. CD44 and CD55 are both overexpressed in HeLa cells, but only moderately expressed in SKBR3 cells. Single-cell spectra were acquired through our whole-cell Raman micro-spectroscopy (see FIG. 14D). As expected, HeLa cell exhibits a higher Raman intensity for both CD44 and CD55 proteins, which correlates well with the expression level of surface proteins (see FIG. 14E). A two-dimensional scatter plot of individual cells, based on the quantitative readouts of two protein channels, present two clearly separable clusters with certain spread each (see FIG. 14F), which not only indicates distinct surface receptor profiles between HeLa and SKBR3 but also suggests expression heterogeneity of individual cells within each cell type.

Multiple cell surface proteins were simultaneously profile on SKBR3 with a cocktail of functionalized Rdots probe. Seven-colored 40 nm Rdots were conjugated with recognition molecules (primary antibodies or aptamers) against nucleolin (Rdot2218-Nucleolin), EpCAM (Rdot2194-EpCAM), MUC1 (Rdot2175-MUC1), CD55 (Rdot2153-CD55), EGFR (Rdot2133-EGFR), CD44 (Rdot2079-CD44), HER2 (Rdot2052-HER2), respectively. Among them, nucleolin, EpCAM, MUC1, and HER2 were stained via aptamers and the others were by antibodies. Firstly, the specificity and semi-quantitative measurement of Rdots probes were individually confirmed, where the Raman signals were found to be correlated with the expression level of surface proteins. After these individual validations, the seven functionalized Rdots were pooled into a cocktail and stained the corresponding surface proteins simultaneously. Seven resolvable Raman peaks were clearly observed, and could be assigned to seven surface proteins, respectively (FIG. 14G, in view in FIG. 14H). Thus, a panel of Rdots-functionalized antibodies and aptamers were created to profile multiplex surface proteins on live cells.

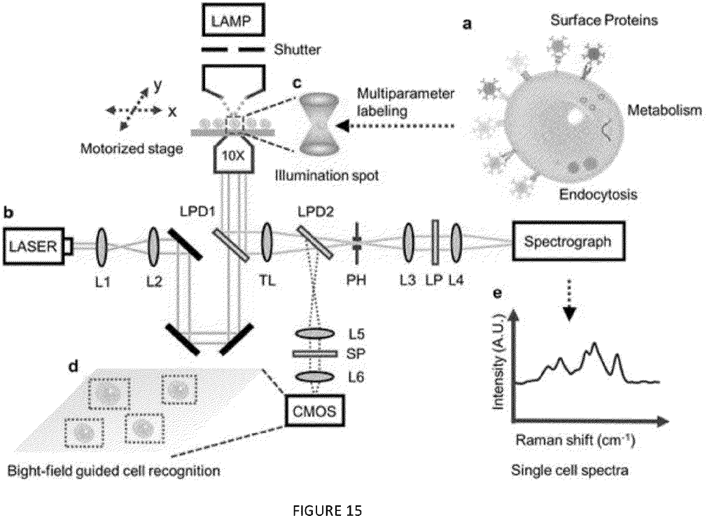

FIG. 15 shows an exemplary schematic diagram of the exemplary automatic confocal micro-Raman system according to another exemplary embodiment of the present disclosure which also illustrates an exemplary workflow for automated whole-cell multiparameter measurement. Portion a of FIG. 15 shows 14 exemplary Raman probes were incorporated to simultaneously quantify cell surface proteins, endocytosis activities, and metabolic dynamics of an individual live cell. Portion b of FIG. 15 shows exemplary home-built whole-cell confocal Raman micro-spectroscopy can excite and detect all Raman probes simultaneously with a single 532 nm laser and an EMCCD detector. Portion c of FIG. 15 shows exemplary back aperture of the objective, which is underfilled, to expand the illumination volume (~8 µm in diameter) for the whole-cell excitation. Portion d of FIG. 15 shows an exemplary brightfield image which was taken to identify and locate the cells in the field of view. Then, the computer-controlled motorized stage moved the slide so that the illumination spot was parked on the cell. Portion e of FIG. 15 shows exemplary single-cell Raman spectra which were acquired with the automatic confocal micro-Raman system. The exemplary components shown in FIG. 15 can be the same as or similar to the exemplary components illustrated in FIG. 1A. For example, L1-L6 can be Lens; LPD1 and LPD2 can be long-pass dichroic beamsplitters; TL can be a tube lens; PH can be a Pinhole; LP can be a long-pass filter; SP can be a short-pass filter.

### Exemplary Endocytic Pathway Profiling by Rdots of Various Sizes and Colors

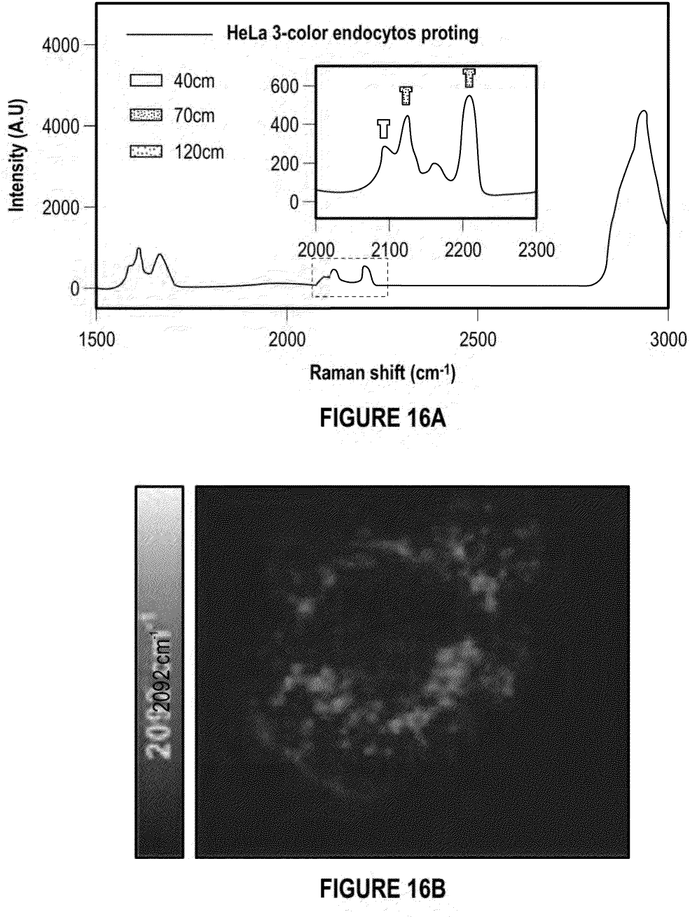

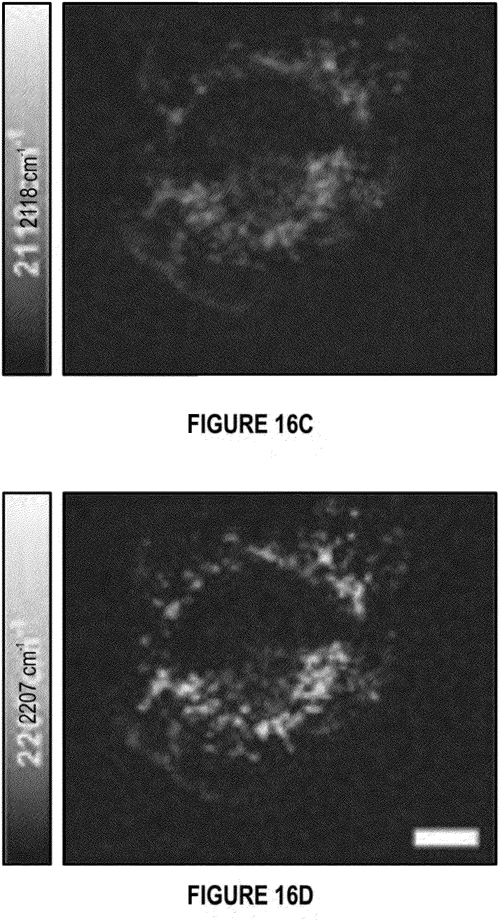

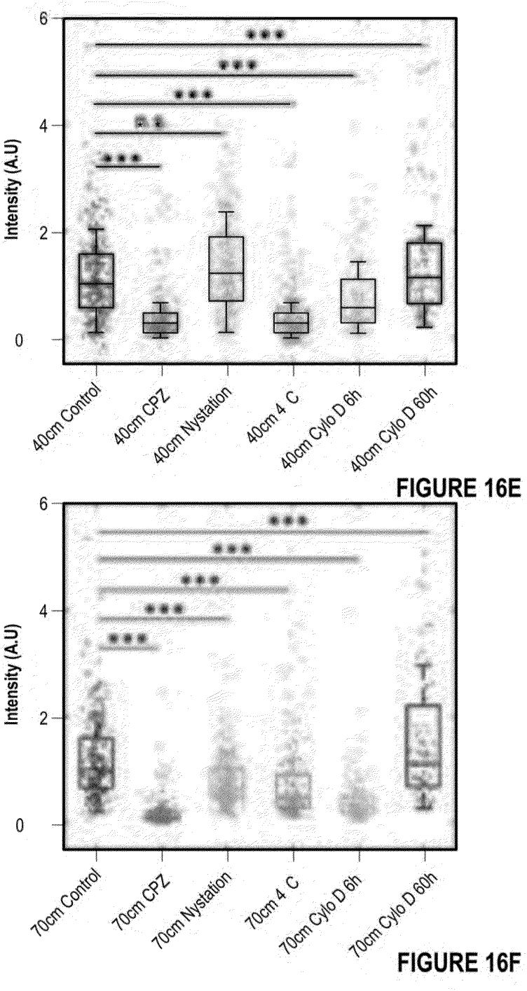

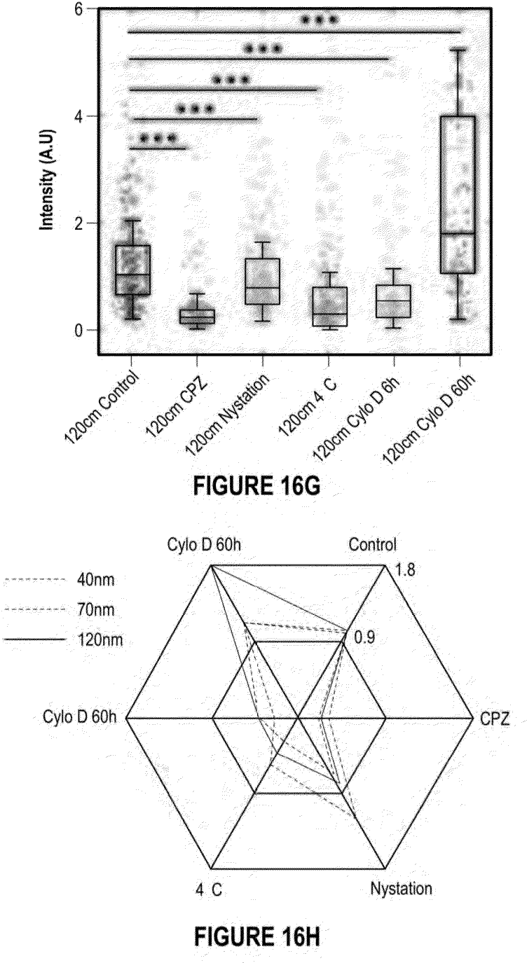

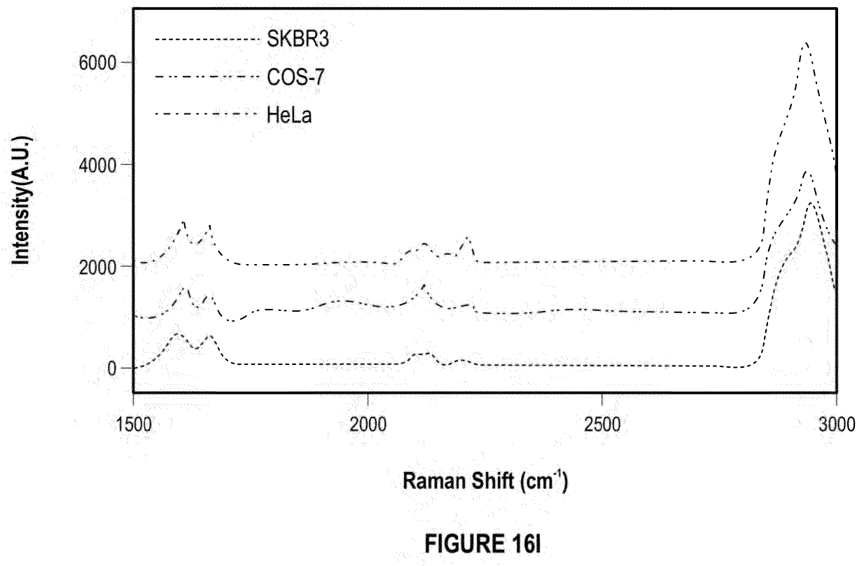

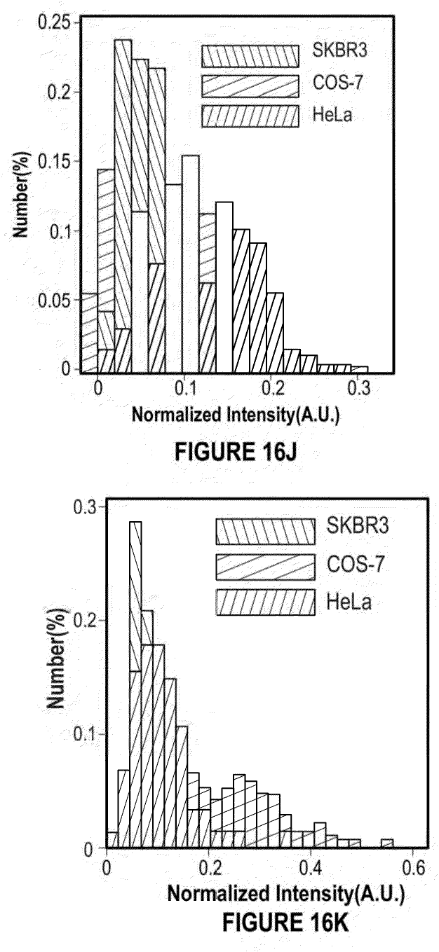

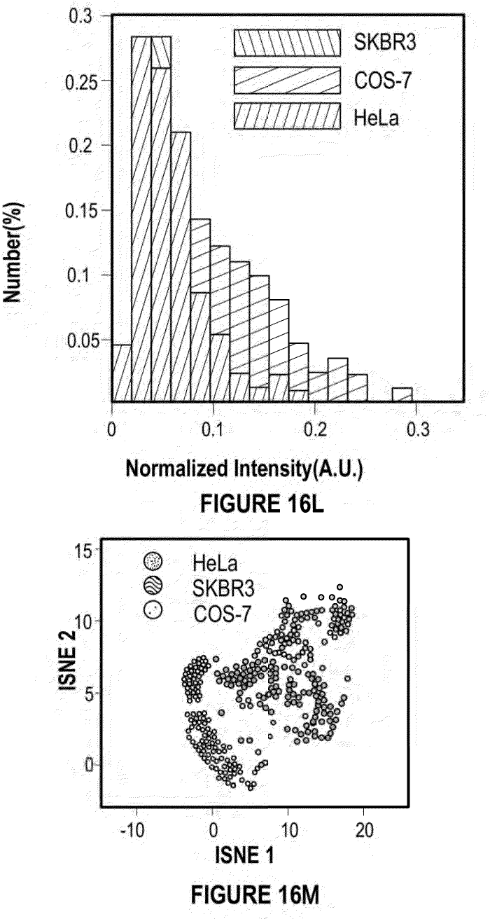

Upon exposure to mammalian cells, PS beads have been reported to enter cells through endocytosis, subsequently reaching the endosomal components. (See, e.g., Reference A40). Although some of the endocytic pathways have been well established, their exact biological roles are still under investigation. (See, e.g., References A41, A42). Intriguingly, it has been suggested that the mechanisms and pathways by which the nanoparticles were internalized are strongly dependent on the size of particles. (See, e.g., Reference A43). Motivated by the independent size and color tunability of Rdots, cellular endocytosis pathways were profiled by incubating cells with Rdots of different sizes and colors. Rdots of 40 nm (Rdot2207), 70 nm (Rdot2118), and 120 nm (Rdot2092) in diameter were incubated with HeLa cells, and their Raman peaks are designed to be mutually resolvable. Indeed, after 6 hours, HeLa cells exhibit three resolvable Raman peaks in the cell-silent window, indicating cellular uptake for each Rdots (FIG. 16A). The spatial distribution of three endocytic Rdots visualized by SRS indicates that Rdots of different sizes all tend to accumulate in the perinuclear region (FIGS. 16B-16D).

For whole-cell Raman acquisition, the corresponding Raman intensity is proportional to the number of internalized Rdots, since the Raman intensity of Rdots remains stable after exposed to cellular microenvironments. We therefore quantified the peak intensity to profile the differential mechanism of Rdots internalization. To this end, cells were first pre-treated with one of three inhibitors, chlorpromazine (CPZ), nystatin, and cytochalasin D (Cyto D). CPZ is a cationic amphiphilic drug used to block clathrin-mediated endocytosis. (See, e.g., Reference A44). Nystatin can decompose cholesterol and inhibit caveolae-mediated endocytosis. (See, e.g., Reference A45). Cyto D inhibits the polymerization of actin filaments, which is required for receptor-mediated endocytosis in mammalian cells. (See, e.g., Reference A46). Additionally, cells were incubated at 4° C. to inhibit energy-dependent endocytosis. Then endocytosis was examined by the quantification of internalized Rdots at the single-cell level. A distinctive response of Rdots internalization was detected when treated with various inhibitors (FIGS. 16E-G), and the radar plot was presented to highlight the fold change after inhibition (FIG. 16H). A similar decreased pattern was observed for Rdots uptake by cells pre-treated with CPZ, implying that their entry into the cells are all sensitive to the assembly of clathrin-coated pits. (See, e.g., Reference A47). In contrast, for nystatin-treated cells, the uptake of Rdots decreased as size increases. The internalization of 40 nm Rdots was unaltered or even slightly higher, while 70 nm and 120 nm Rdots were both inhibited, indicating that caveolae receptors are preferentially involved in the internalization of larger Rdots. (See, e.g., References A48, A49). Consistent with blocked endocytosis at low temperature, (see, e.g., Reference A49), exposure of cells to a low temperature significantly reduced the uptake of all three Rdots. Rdots internalization is also suppressed by short-term Cyto D incubation, implying that actin filaments are necessary for efficient endocytosis. Interestingly, after incubation with Cyto D for 60 hours, the cellular uptake of Rdots was markedly promoted, likely due to apoptosis-induced membrane permeability alteration. Therefore, the results of inhibition treatments suggest that different particle sizes do differ during endocytosis.

This exemplary procedure was demonstrated in evaluating endocytosis differences in HeLa, SKBR3, and COS-7 cell lines. After pre-treated with three-color/size endocytic Rdots for 6 hours, single-cell Raman spectra were acquired. The spectral feature for three cell lines appears distinct in the cell-silent region, suggesting different endocytic performances (Figure 161). Quantitative readouts of three endocytic Rdots were then analyzed and histograms were constructed from single cells (FIGS. 16J-L). Histogram from a single-type Rdot (either 40 nm or 70 nm or 120 nm) alone has less discriminate power to distinguish between HeLa, SKBR3, and COS-7 cells, largely because of the wide distributions from cell-to-cell variation. To harness information from all three endocytic Rdots, a multiparameter-based t-distributed stochastic neighbor embedding (t-SNE) (see, e.g., Reference A50) procedure was employed, and three clusters were identified (FIG. 16M), suggesting that endocytosis could serve as an informative dimension of cell phenotyping. This success showcased the great potential of endocytic profiling in cell-type identification, as well as the advantage of employing endocytic Rdots of different sizes.

FIGS. 16A-16M show exemplary illustration of an exemplary endocytic profiling assay by multi-color Rdots. In particular, FIG. 16A shows exemplary spontaneous Raman spectra of HeLa cells acquired after incubated with Rdot2207 (40 nm), Rdot2118 (70 nm), and Rdot2092 (120 nm) for 6 hours, averaged over 128 individual cells. The peak position of each Rdots was indicated by the arrow. Excitation: 532 nm (400 mW), acquisition time: 1 s. FIGS. 16B-16D show exemplary SRS images of cellular distribution for three endocytic Rdots, (B) 40 nm (C) 70 nm (D) 120 nm, scale bar: 10 µm. FIGS. 16E-16G show exemplary box plots illustrating the normalized Raman intensity distribution of three endocytic Rdots under the stress of clathrin inhibitor CPZ and caveolae inhibitor nystatin, low temperature and actin inhibitor Cyto D (incubated for 6 hours and 60 hours), (FIG. 16E - 40 nm, FIG. 16F - 70 nm, and FIG. 16G - 120 nm. N_{control} = 309; N_{CPZ} = 209; N_{nystatin} = 387; N_{4°C} = 255; N_{Cyto} _{D} ₆ₕ= 198; N_{Cyto} _{D} ₆₀ₕ= 87. For example, *** denotes a significant difference between the two groups (P<0.001). n.s. denotes not significant (P>0.05). FIG. 16H shows an exemplary illustration providing exemplary properties of inhibition displaying on Radar diagrams and each inhibition action is provided with an axis. FIG. 16I shows exemplary averaged spontaneous Raman spectra of HeLa, SKBR3, and COS-7 cells acquired after incubated with three endocytic Rdots for 6 hours, N_{HeLa}=128; N_{SKBR3}=134; N_{COS-7}=130. Excitation: 532 nm (400 mW), acquisition time: 1 s. FIGS. 16J-L show exemplary histogram showing the Raman intensity distribution of three endocytic Rdots uptake by HeLa, SKBR3, and COS-7 cells, FIG. 16J - 40 nm, FIG. 16K - 70 nm, and FIG. 16L - 120 nm. FIG. 16M shows an exemplary three-color endocytic profiling of HeLa and SKBR3 and COS-7 cells projected on t-SNE plot, colored by cell types.

### Exemplary Development of 14-Plexed Live-Cell Raman Profiling Platform

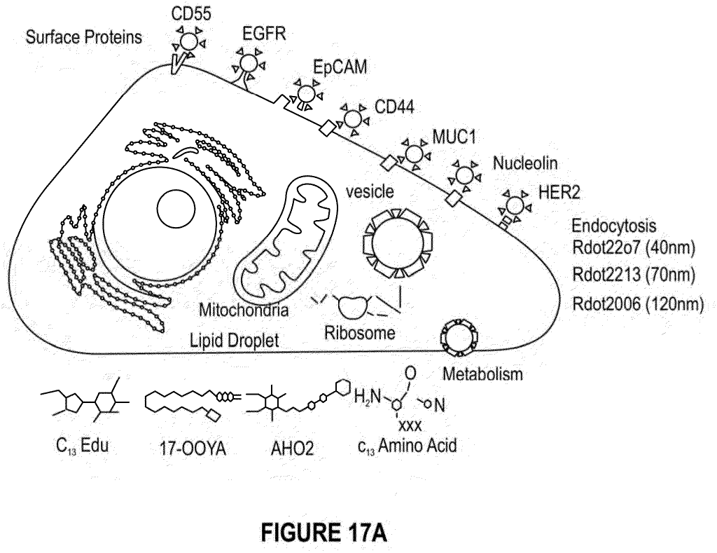

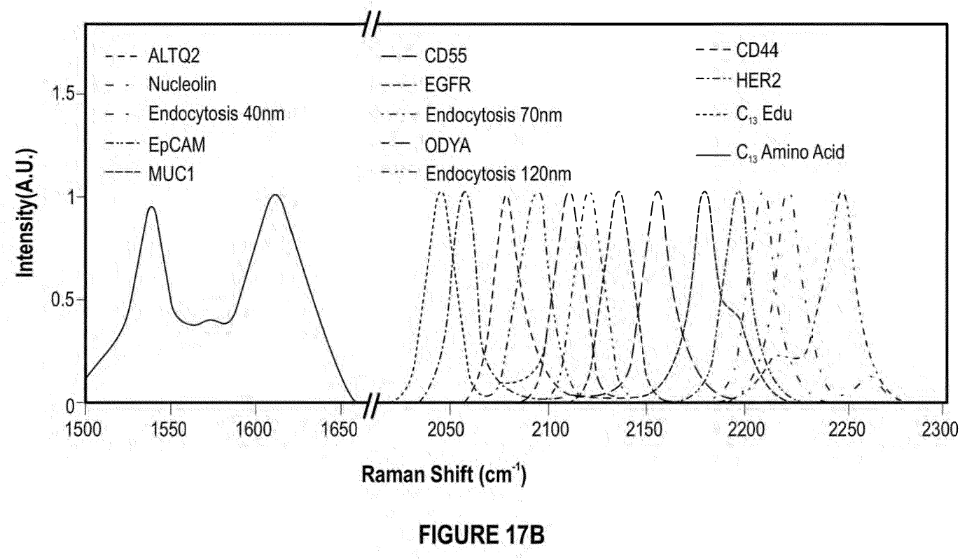

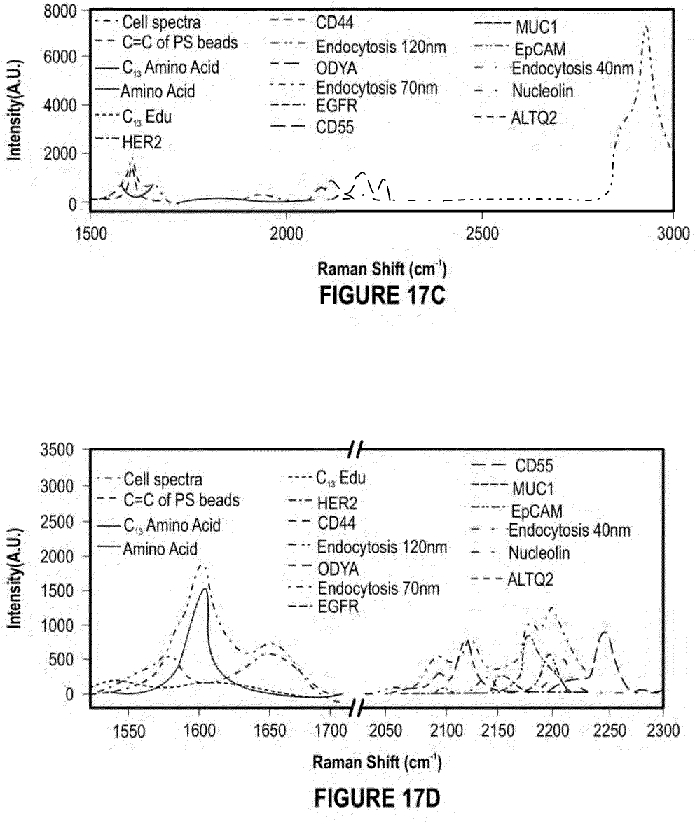

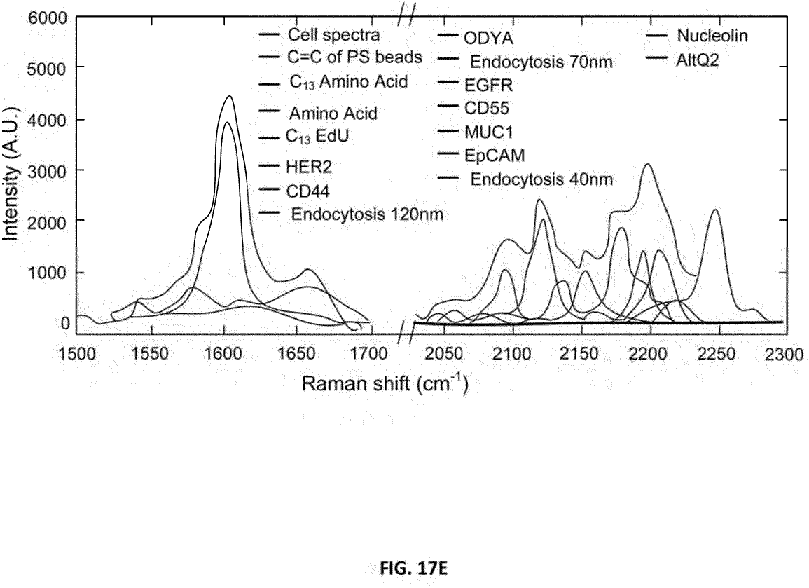

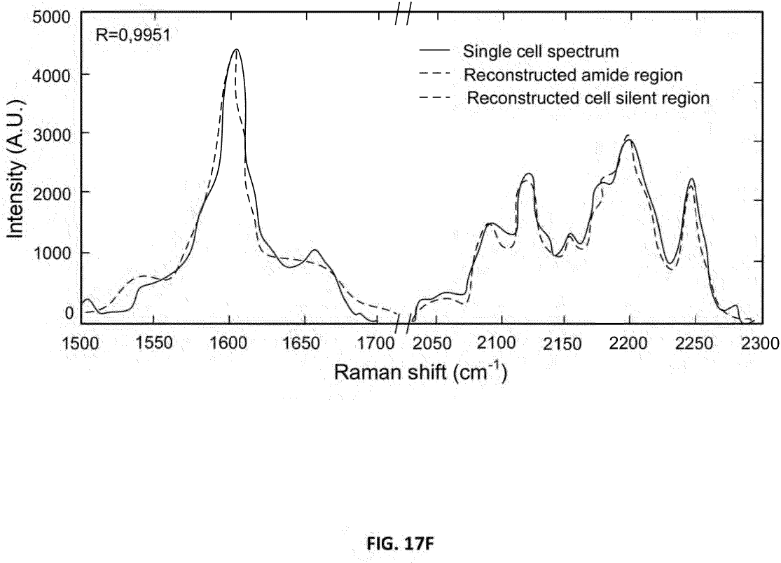

FIGS. 17A-17F show exemplary illustrations providing exemplary principle and implementation of multiparameter Raman profiling of individual living cells, according to an exemplary embodiment of the present disclosure. For example, FIG. 17A shows an exemplary diagram illustrating the cellular localization of 14 Raman probes for multiparameter live-cell profiling. FIG. 17B shows a graph of exemplary normalized Raman peaks of 14 Raman probes with distinct frequencies. FIG. 17C shows a graph of automated whole-cell Raman microscope. FIGS. 17C and 17D show graphs exemplary 14-plexed live-cell Raman spectra with unmixing processing (see FIG. 17C) and zoom-in view of cell amide and silent window (see FIG. 17D), averaged over 213 individual cells. FIGS. 17E and 17F show graphs of exemplary zoom-in view of single live-cell Raman spectrum with unmixing processing (see FIG. 17E) and reconstructed amide and cell silent region (see FIG. 17F), the Pearson correlation efficient between reconstructed and original spectra R =0.9951.

In particular, As indicated herein, seven-colored Rdots were developed to evaluate cell surface proteins (nucleolin, EpCAM, MUC1, CD55, EGFR, CD44, and HER2) and three-colored Rdots (Rdot2207, Rdot2118, and Rdot2092) to probe cellular endocytosis in live cells. No significant signs of cytotoxicity were observed during Rdots staining and endocytic test, supporting their feasibility in the live-cell assay. To provide a more comprehensive indication of phenotypic diversity of single cells, four additional metabolic probes, ¹³C-EdU, 17-Octadecynoic Acid (17-ODYA), diyne-tagged CoQ analogues AltQ2 (see, e.g., Reference A35) and ¹³C-amino acids (¹³C-AA), were incorporated to enrich the Raman probe panel (see FIG. 17A). Under the substitution of ¹³C-AA, the Raman peak at 1574 cm⁻¹ and 1659 cm⁻¹, ascribed to cellular amide II and amide I, will shift to lower wavenumber by about 40 cm⁻¹, as a result of the ¹³C containing amide bonds in the newly synthesized protein. (See, e.g., Reference A51). The metabolic dynamics of fatty acids and DNA synthesis were probed by alkyne-tagged fatty acid 17-ODYA and ¹³C-EdU (see, e.g., References A52, A53), respectively. The electron transport in the mitochondria respiratory chain was characterized by CoQ analogues AltQ2. (See, e.g., Reference A35). These metabolic activities of small metabolites are difficult for fluorescence methods. (See, e.g., Reference A16). In total, 14 Raman probes were included in our multiplexed panel (see FIG. 17B).

For example, almost all of the 14 probes show individual narrow Raman peaks at the cell-silent window, enabling highly sensitive detection in a background-free manner. Meanwhile, we designed them so that their Raman peaks can be mutually separated from each other. In contrast, such a resolvability would be challenging for fluorescence microscopy with broad emission peaks. To acquire, e.g., the 14-plex Raman information all together, an exemplary whole-cell confocal Raman micro-spectroscopy can be implemented which can excite and detect all Raman probes simultaneously with a single 532 nm laser source and an EMCCD detector (see, e.g., Reference A38) (see FIG. 17C). Equipped with fully automated hardware and brightfield-guided cell identification and localization procedures, efficient acquisition was achieved for single-cell readouts with a throughput of 3600 cells per hour (1 s per cell). Compared with fluorescence-based flow cytometry which requires multiple lasers and detectors and cumbersome spectral compensation, the exemplary Raman instrument according to the exemplary embodiments of the present disclosure as described herein is believed to be straightforward, reliable, and cost-effective.

An exemplary 14-plex Raman probe profiling cocktail was demonstrated under whole-cell Raman micro-spectroscopy. Metabolic probes including ¹³C-EdU, 17-ODYA, and ¹³C-AA were first supplemented to cell culture media for 60 hours to report various metabolic activities. For endocytic profiling, cells were subsequently incubated with three Rdots for 6 hours. Then, seven functionalized Rdots cocktail was pooled to stain cell surface proteins simultaneously. Prior to the spectral acquisition, AltQ2 probe was added to live cells. Single-cell Raman spectra of SKBR3 cells were acquired with 1 second acquisition time (see FIG. 17C). To decipher the contribution of individual Raman probes, the amide region (1500 cm⁻¹-1700 cm-¹) and cell-silent region (2000 cm⁻¹ - 2300 cm-¹) were linearly decomposed by using the reference spectral profiles of each probe (see FIG. 17D) (See, e.g., References A54-A56). The unmixing procedure was applied on single-cell Raman spectra (see FIG. 17E). The spectrum reconstructed from unmixed components is highly consistent with the single-cell spectrum measured originally (see FIG. 17F). The decomposed peak intensity for the Raman probe with relatively low SNR still coincides well with the ground truth value, indicating a reliable retrieval after unmixing. Taken together, an exemplary robust Raman-based platform is established to provide a highly multiplexed profiling of live cells. This represents a high Raman-based multiplexing, going beyond the previous record of 8-plex tissue imaging and 10-color live-cell imaging in the literature. (See, e.g., References A25, A26).

### Exemplary Live-Cell Multiparameter Profiling Differentiates Various Drug Actions

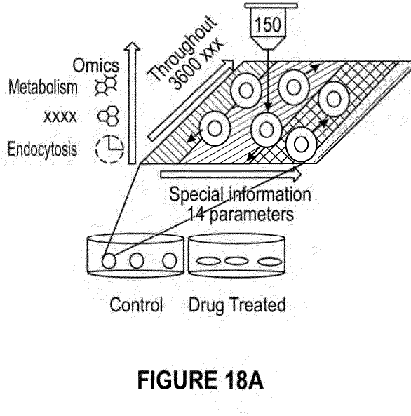

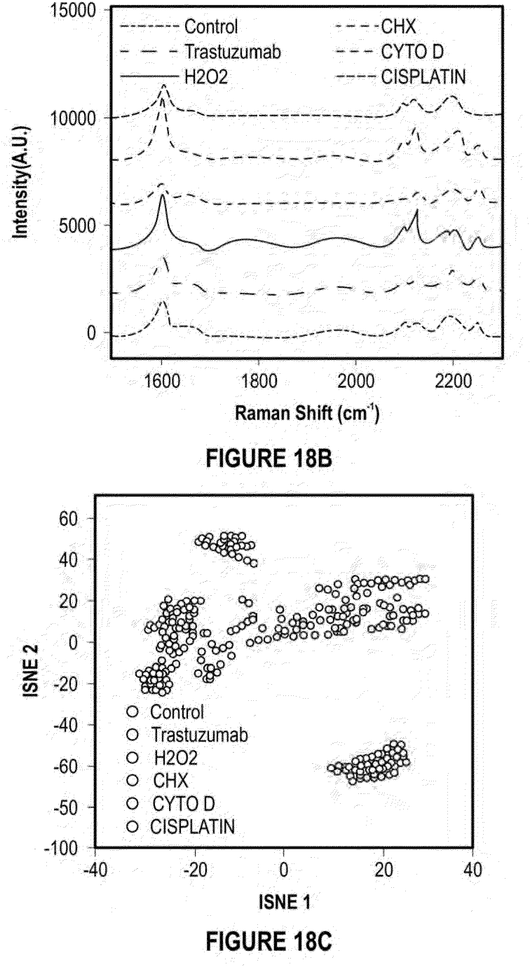

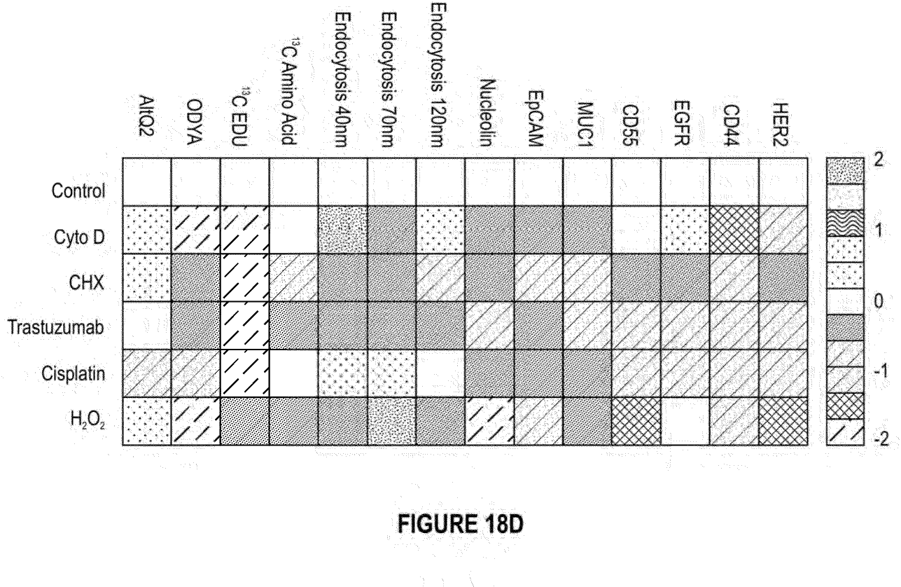

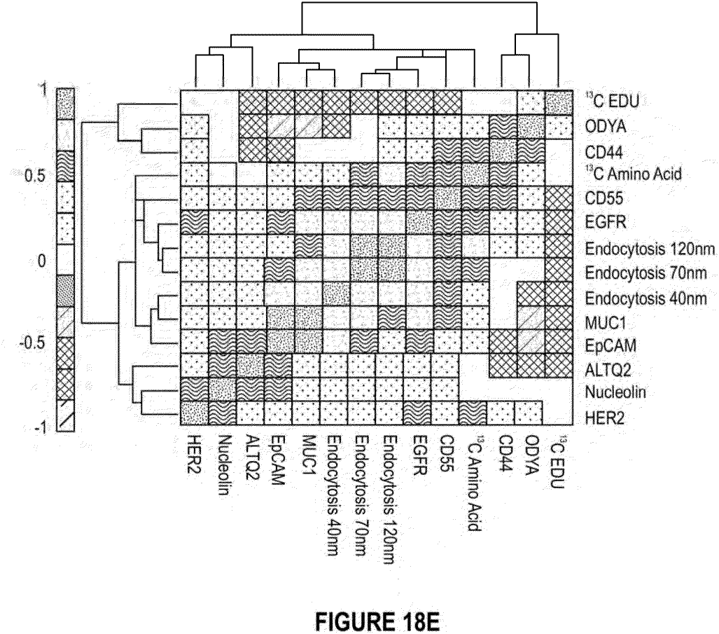

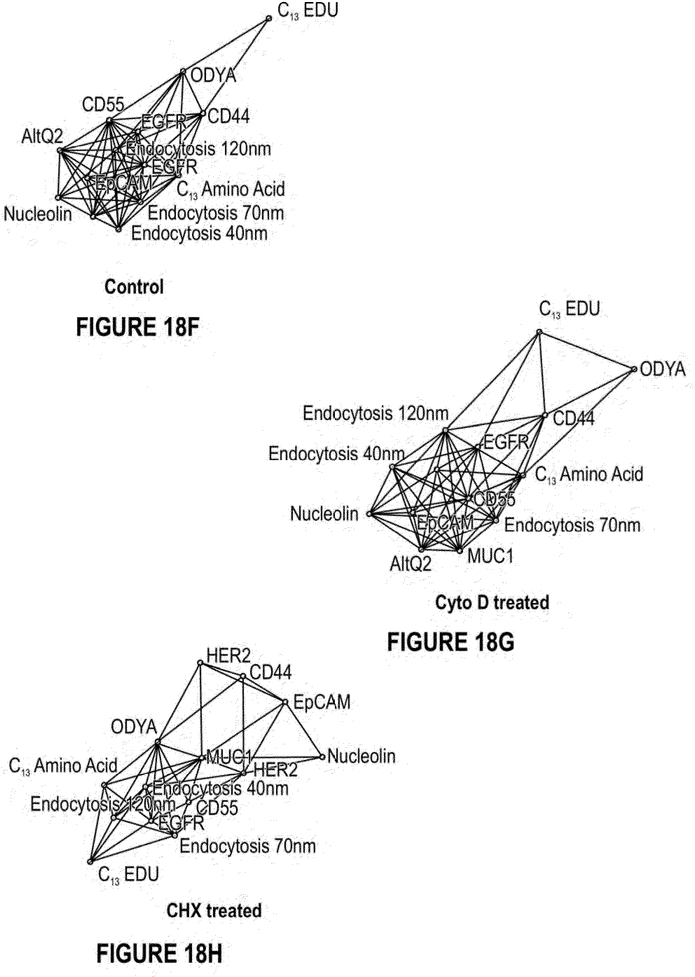

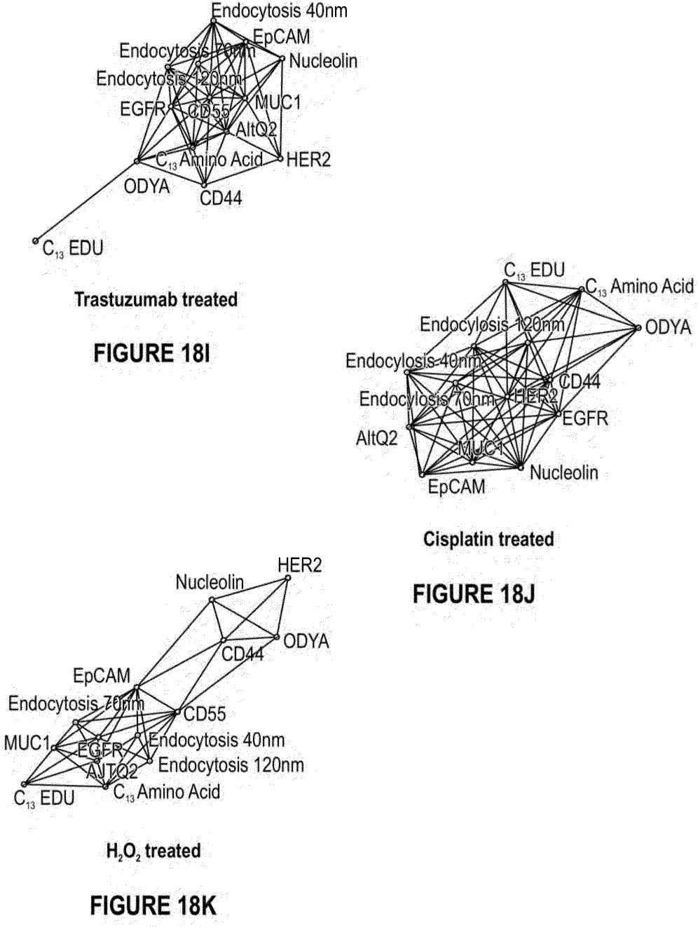

Cell-based profiling techniques are increasingly being used in drug discovery to monitor cell response upon drug treatment. (See, e.g., Reference A57). Optical techniques have unique advantages by allowing non-invasive, fast, and cost-effective readouts of individual living cells. (See, e.g., Reference A58). However, most high-content profiling assays have been primarily focusing on partial aspects of biological phenotypes. (See, e.g., Reference A59). Integration of different phenotypes will facilitate a more systematic understanding of the mechanism of drug candidates. (See, e.g., Reference A60). To this end, the live-cell multiparameter profiling platform is demonstrated in revealing phenotypic responses upon five chemotherapy reagents (see FIG. 18A). Trastuzumab is a HER2-targeted monoclonal antibody developed for clinical use in breast cancer patients. (See, e.g., Reference A61). Hydrogen peroxide (H₂O₂) is well known as a major member of reactive oxygen species (ROS). (See, e.g., Reference A62). Cycloheximide (CHX) exerts its action by inhibiting protein synthesis. (See, e.g., Reference A63). Cyto D causes the disruption of actin filaments and inhibition of actin polymerization. (See, e.g., Reference A64). Cisplatin is an inhibitor of DNA synthesis and cell growth. (See, e.g., Reference A65). After labeling with 14 Raman probes, single-cell Raman spectra of SKBR3 cells under treatment of five chemotherapy reagents, as well as untreated cells, were acquired through the automated whole-cell Raman micro-spectroscopy (see FIG. 18B).

A single-cell Raman spectra was decomposed to retrieve the intensities of 14 individual probes and constructed a population-averaged heatmap for all 14 phenotypic features (see FIG. 18C). The exemplary results are largely consistent with the prior reports. For instance, Cyto D treatment inhibits both protein and DNA synthesis, consistent with previous findings. (See, e.g., References A66, A67). Long-term Cyto D treatment can promote cellular internalization of Rdots, consistent with our demonstration above (see FIG. 16H). Previous research has shown that inhibition of actin polymerization, such as by Cyto D, suppresses CD44 surface expression. (See, e.g., Reference A68), which is also agreed with the profiling results, further proving the reliability and capability of the drug action study. CHX can interfere with the process other than protein synthesis such as DNA synthesis by multiple direct and indirect mechanisms, (see, e.g., Reference A69), which has been captured by the ¹³C-EdU and ¹³C-AA probes. For H₂O₂ treated cells, the intermediate ROS generated by H₂O₂ is potent oxidants of proteins, lipid, and nucleic acids, thus retarding their turnover rate, (see, e.g., Reference A70, A71), consistent with the phenotype revealed by 17-ODYA. The results also revealed new insights. For example, cellular internalization of Rdots of 70 nm and 120 nm was also promoted under H₂O₂ treatment, indicating a membrane permeability change. For another example, increased internalization of AltQ2 was observed after treated with Cyto D, CHX, and H₂O₂, indicating alteration of mitochondrial membrane potential by these chemotherapeutic drugs. (See, e.g., References A72-A75).

FIGS. 18 shows exemplary live-cell multiparameter profiling which reveals cellular response characteristics for different drugs. FIG. 18A shows exemplary schematic illustration of phenotypic profiling over multiple chemotherapy treatments. FIG. 18B shows exemplary averaged spontaneous Raman spectra of SKBR3 cells acquired after drug perturbation and probes incubation, N_{Control}=213; N_{CytoD}=189; N_{CHX}=167; N_{trastuzumab}=235; N_{Cisplatin}=136; N_{H2O2}=267. FIG. 18C shows exemplary population-averaged heatmap for individual parameters which reveals the mechanisms of multiple drug actions. The intensity of each parameter is normalized to the control sample. Dark red denotes increased intensity, dark blue denotes decreased intensity. FIG. 18D shows exemplary t-SNE scatter plot clustered SKBR3 cell populations based on multiparameter profiling results, colored by drug treatments. FIG. 18E shows exemplary unsupervised hierarchical clustering heatmap analysis of Pearson correlation coefficients over 14 parameters for untreated control cells. Dark red denotes high positive correlations, dark blue denotes high negative correlations. FIGS. 18F-18K show exemplary network graphs of correlations among 14 parameters for untreated control cells (F); Cyto D treated cells (see FIG. 18G); CHX treated cells (see FIG. 18H); Trastuzumab treated cells (see FIG. 18I); Cisplatin treated cells (J); H₂O₂ treated cells (see FIG. 18K). All nodes can be connected and the length of the edges connecting two nodes can represent the inverse of correlation degree between two parameters.

### Exemplary Single-Cell Measurement Enables Clustering, Correlation, and Network Analysis

One of many advantages of single-cell measurement over the conventional population-averaged one can be due to the detailed information from a large number of individual cells. This is especially important for highly multiplexed readouts, as extensively documented in single-cell transcriptomics. (See, e.g., References A76-A78). The exemplary single-cell data according to the exemplary embodiment of the present disclosure can facilitate a performance of clustering, correlation, and/or network analysis, which can likely be beyond the population-averaged results. For clustering analysis, a two-dimensional t-SNE plot was constructed to process the high-dimensional Raman spectral readouts from all the cells (see FIG. 18D). It is intriguing that cells treated by different chemotherapy reagents form clear clusters in the t-SNE space, highlighting distinct phenotypic responses.

Pearson correlation coefficients can be computed between any pairs of 14 phenotypic features across all the measured cells (see FIG. 18E). Two tumor-associated proteins MUC1 and EpCAM exhibit a highly-positive correlation, thereby suggesting a consistent role in cancer metastatic progression. (See, e.g., Reference A79). Interestingly, a strong correlation was observed over the internalization of three endocytic Rdots, likely due to that they are probing the same class of biological process. The expression of transmembrane receptor EGFR is positively correlated with endocytic channels, which is reasonable since endocytosis plays an important role in EGFR-mediated cell signaling. (See, e.g., Reference A80).

The correlation network of 14 phenotypic features were analyzed to reveal a systematic co-regulation and interaction across cell metabolites, endocytosis, and surface proteins (see FIGS. 18F-18K). For untreated cells, HER2, EpCAM, EGFR, and 120 nm endocytosis are associated with the most other phenotypic features, thereby acting as central nodes of the network (see FIG. 18F). According to the “centrality-lethality rule”, these nodes tend to be essential in biological networks and more likely to be identified as treatment targets. (See, e.g., Reference A81). In contrast, metabolic probes ODYA and ¹³C-EdU locate away from the primary cluster, suggesting that those nodes might work independently. Analysis of the drug-induced network topology can facilitate understanding of drug responses and accelerate drug development. (See, e.g., Reference A82). Interestingly, different network topologies emerged over drug perturbations. For example, after CHX treatment, metabolites nodes tend to approach the cluster center (see FIG. 18H), indicating that metabolites form more efficient feedbacks to drive cells returning to normal state. (See, e.g., Reference A83). H₂O₂ treated cells exhibit two neighboring network modules connected by the bridge nodes (CD55 and EpCAM, FIG. 18K). The independent regulation of both modules from those bridge nodes makes them attractive as drug targets. (See, e.g., Reference A83). This exemplary observation can further assist in exploring the appropriate drug combinations and multi-target drugs. (See, e.g., Reference A84). Taken together, the rich information extractable from single-cell multiparameter profiling holds great potential in, among others, unraveling complex interactions between multiple molecules for predicting uncertain drug mechanisms.

### Exemplary Cell Culture

HeLa, SKBR3, and COS-7 cells were all obtained from the American Type Culture Collection (ATCC) and kept under standard cell culture conditions (5% CO₂, 37° C.). HeLa and COS-7 cells were cultured in DMEM media (Gibco, 11965118) supplemented with 10% fetal bovine serum (FBS, Gibco, 10099141) and 1 % penicillin/streptomycin (P/S, Gibco, 15140148). SKBR3 cells were cultured in McCoy’s 5A media (Gibco, 16600082) supplemented with 10% FBS and 1% P/S.

### Exemplary Rdots Preparation and Bioconjugation

Incorporation of Carbow dyes was achieved by swelling the 4% w/v polystyrene (PS) beads (Invitrogen, C37232, C37233, C37479) in a solvent mixture containing 160 µL 4% w/v PS beads, 160 µL reverse osmosis (RO) water and 120 µL Tetrahydrofuran (THF, Sigma, 401757)), and by adding a controlled amount of Carbow dyes to the mixture (refer to Table 1 for specific dye concentration). After 30 minutes of gentle agitation at room temperature (RT), 2 mL 20 mM phosphate buffer (PH 7.3) was subsequently added to shrink the Rdots. Excess dyes were removed by three rounds of centrifugation and resuspension in RO water using 30 K MWCO filters (Millipore, UFC9030). Rdots bioconjugation to antibodies and aptamers were carried out through carboxyl-to-amine crosslinking using the ethyl dimethylaminopropyl carbodiimide (EDC, Thermo Scientific, 22980) and sulfo-NHS (Sigma, 56485). To activate the carboxyl groups on beads surface for covalent conjugation, 200 µL 4% w/v beads were mixed vigorously with 100 µL 100 mg/mL freshly prepared EDC solution and 100 µL 150 mg/mL sulfo-NHS in MES buffer (25 mM, PH 6.0) at RT for 30 minutes. Excess EDC and sulfo-NHS were separated by two rounds of centrifugation (16000 rmp) and resuspension in RO water using 30 K MWCO filters (Millipore, UFC503024). The purified beads with activated carboxyl groups were then exposed to 200 µL 35 mM NH2-PEG-COOH (Laysan Bio Inc, NC1641410) and 200 µL 320 mM NH2-PEG-OH (Laysan Bio Inc, NC1641409) in DPBS buffer (PH 8.1) for 3 hours at RT to yield a well-shielded PEG layer. Excess PEG molecules were removed by three rounds of centrifugation and resuspension in RO water using 100 K MWCO filters (Millipore, UFC510024). For antibody conjugation, the carboxyl groups on PEGylated beads were then activated with 100 µL 100 mg/mL freshly prepared EDC solution and 100 µL 150 mg/mL sulfo-NHS in MES buffer (25 mM, PH 6.0) at RT for 30 minutes. After two rounds of centrifugation, the activated beads were then mixed with antibodies at a bead: antibody molar ratio of 1:30 and react for 3 hours in HEPES buffer (10 mM, PH 8.3) at RT. The bead-Ab conjugates were separated from free antibodies by centrifugation for 3 rounds at 16000 rpm. Then the conjugates were resuspended in DPBS buffer (Gibco, 14190136) for use. For aptamer conjugation, the activated beads were mixed with aptamer at a bead: aptamer molar ratio of 1:100 and react for 3 hours in HEPES buffer (10 mM, PH 8.3) at RT. Free aptamers were removed by four rounds of centrifugation with 100 K MWCO filters and resuspension in DPBS buffer for use (see Table 4 for antibody catalog numbers and aptamer sequences).

TABLE 3

The concentration of incorporated Carbow dyes

| Dye | Carbo | Carbo | Carbo | Carbo | Carbo | Carbo | Carbo | Carbo | Carbo | Carbo |
|---|---|---|---|---|---|---|---|---|---|---|
| | w | w | w | w | w | w | w | w | w | w |
| | 2218 | 2207 | 2194 | 2175 | 2153 | 2133 | 2118 | 2092 | 2079 | 2052 |
| Concentration | 10 | 10 | 10 | 2.5 | 2.5 | 1 mM | 1 mM | 1 mM | 1 mM | 1 mM |
| | mM | mM | mM | mM | mM | | | | | |

TABLE 4

Antibodies and aptamers used for cell surface protein staining

| | |
|---|---|
| Antibody/ Aptamer | Catalog numbers/ sequences |
| Mouse anti-human CD55 Clone: 28 | Invitrogen MA5-29118 |
| Rat anti-human CD44 Clone: IM7 | Invitrogen 14044185 |
| Mouse anti-human EGFR Clone: 528 | Bio X Cell BE0279R005MG |
| Nucleolin aptamer (see, e.g., Ref. A14) | /5AmMC6/TTTTT1GGTGGTGGTGGTTGTGGTGGTGGTGG [SEQ ID NO: 1] |
| MUC1 aptamer (see, e.g., Ref. A87) | /5AmMC6/TTTTTTGCAGTTGATCCTTTGGATACCCTGG [SEQ ID NO: 2] |
| HER2 aptamer (see, e.g., Ref. A88) | /5AmMC6/TTTTTTAACCGCCCAAATCCCTAAGAGTCTGCACTTGTCATTTTGTATATGTATTTGGTTTTTGGCTCTCACAGACACACTACACACGCACA [SEQ ID NO: 3] |
| EpCAM aptamer (see, e.g., Ref. A88) | /5AmMC6/TTTTTTCACTACAGAGGTTGCGTCTGTCCCACGTTGTCATGGGGGGTTGGCCTG [SEQ ID NO: 4] |

### Exemplary pH Stability of Rdots

To assess the effect of pH conditions on Rdots stability, the Rdots were exposed to opti-MEM (Gibco, 31985062) with pH 7.0, 5.5, and 4.5. The pH of opti-MEM was adjusted by adding acid dropwise with constant stirring. The time-dependent Raman intensity was measured through our home-built Raman microscope.

### Exemplary Metabolic Probe Preparation

To prepare ¹³C-AA DMEM, 4 mg/mL algae ¹³C amino acid mix (CLM-1548, Cambridge isotope) was dissolved in RO water supplemented with 10% FBS, 1% P/S and other components including vitamin, inorganic salts, and glucose according to DMEM media formula (Invitrogen, 11965). 17-ODYA (Tocris Bioscience, 06-171-0) was dissolved in DMSO and a working stock solution of 4 mM was prepared by 1:6 complexing to BSA (Sigma-Aldrich, A6003). ¹³C-EdU was synthesized as the previous reported. (See, e.g., Reference A53). AltQ2 (a generous gift from Professor Mikiko Sodeoka) was dissolved in DMSO to get a stock solution of 10 mM for use.

### Exemplary Cell Proliferation Assay

To evaluate the cytotoxicity of Rdots on cells, SKBR3 cells were incubated with 1 nM endocytosis beads for 6 hours and then mixed with 10 nM Rdots for surface protein labeling. Cell viability was studied using Live/Dead cell double staining by incubating with 2 µM calcein-AM (Invitrogen, C3099) and 2.5 µM propidium iodide (PI, Sigma-Aldrich, P4864) for 30 minutes at 37° C. Fluorescent images were acquired by Olympus confocal microscopy prior to viable/dead cell counting.

### Exemplary Live Cell Surface Protein Staining

Cells were dissociated using trypsin-EDTA (Gibco, 25-200-056) on reaching 75% confluence and then harvested in the tube. After two rounds of washing with ice-cold DPBS, cells were resuspended on DPBS buffer with 5 mM MgCl₂, 1 mg/mL yeast tRNA (Invitrogen, AM7119), and 1% BSA (Sigma-Aldrich, 05470) to reach 10⁷ cells per mL. For aptamer annealing, aptamers conjugated Rdots were incubated on a heat block at 90° C. for 4 minutes and then slowly cool to RT. Then cells were stained with seven-colored Rdot conjugates at a concentration of 10 nM for 30 minutes on ice. Followed by three rounds of washing with DBS buffer with 5 mM MgCl₂ and 1% BSA, cells were attached to a poly-L-lysine coated coverslip (Neuvitro GG12PDL) and mounted onto the microscope for Raman measurement.

### Exemplary Automated Raman Spectrometer

The schematic of the home-built Raman microscope is shown as our previously reported. (See, e.g., Reference A38). Here, the 10X objective was underfilled to reach an illumination diameter of 8-10 µm. A motorized stage was installed to automatically park on cells identified through bright filed. The entire system was controlled through a LabVIEW-based software module (National Instrument).

### Exemplary Stimulated Raman Scattering (SRS) Microscopy

The setup of SRS microscopy has been described previously. (See, e.g., Reference A26). Briefly, an integrated laser (Applied Physics and Electronics, Inc., picoEMERALD) was coupled into an inverted laser scanning confocal microscope (Olympus, FV1200). The Stokes beam (1,064 nm, 6 ps pulse width) was intensity-modulated at 8 MHz by electro-optic-modulator, and a tunable pump beam (720-990 nm, 5-6 ps pulse width) was produced by the optical parametric oscillator. The laser beams were focused on the sample through a 25x water immersion objective (Olympus, XLPlan N, 1.05 NA MP). For cellular imaging, 100 mW pump and 400 mW Stokes power were used, with 40 us time constant and the matching pixel dwell time.

### Exemplary Multiparameter Live-Cell Profiling

SKBR3 cells were seeded onto 18 mm round quartz coverslips (Electron Microscopy Sciences, 103302-258) and then maintained in a culture environment for 48 hours to reach 90% confluence. Then the culture medium was replaced with ¹³C-AA DMEM containing 200 µM 17-ODYA and 50 nM ¹³C-EdU for 60 hours. For the drug testing samples, cells were subject to the drug treatment simultaneously (see Table 5 for specific drug concentration). Followed by three rounds of gentle washing of DPBS buffer, cells were subsequently incubated with three-colored endocytic Rdots at 1 nM in serum-free DMEM medium. 6 hours later, cell surface markers were stained with seven-colored Rdots at a concentration of 10 nM for 1 hour on ice. After that, cells were washed extensively with DPBS buffer with 5 mM MgCl₂ and 1% BSA. Then cells were rinsed with DPBS buffer with 5 mM MgCl₂ and 40 µM AltQ2 before mounting onto the microscope for Raman measurement. Spectral unmixing was performed through the Least Square Method (LSM).

TABLE 5

Chemotherapy reagents mechanism and concentration tested in this study

| | | |
|---|---|---|
| Chemotherapy agents | Mechanism of action | Concentration |
| Cisplatin | DNA synthesis inhibitor | 0.2 µg/mL |
| Cycloheximide | Protein synthesis inhibitor | 1 µg/mL |
| Trastuzumab | Monoclonal antibody targeting HER2 | 100 µg/mL |
| Cytochalasin D | Actin inhibitor | 2.5 µg/mL |
| H₂O₂ | chemical oxidation of cellular components | 100 µM |

### Exemplary Spectral Unmixing Processing

To decipher the contribution of individual Raman probes, spectra unmixing was performed for the amide region and cell silent region, respectively. The normalized spectra of 16 components (including 14-plexed Raman probes, C=C vibration of PS bead, and ¹²C amide peak) in FIG. 18E were employed as reference spectra and characterized by library M_{amide} and Mₛᵢₗₑₙₜ, respectively. After background removal, the trimmed single-cell Raman spectrum I_{amide} (1500 cm⁻¹ - 1700 cm-¹) and Iₛᵢₗₑₙₜ (2000 cm⁻¹ - 2300 cm-¹) can be deconvolved into the weighted (θ) sum of reference spectrum and noise N. The process of linear unmixing can be described as follows:

$${\text{I}}_{\text{silent}} = {\text{M}}_{\text{silent}} \times {θ}_{\text{silent}} + \text{N;}$$

$${\text{I}}_{\text{amide}} = {\text{M}}_{\text{amide}} \times {θ}_{\text{amide}} + \text{N;}$$

Approximating that the value of N is negligible for the low-noise spectrum, so here we made an estimation:

$${\text{I}}_{\text{silent}} \approx {\text{M}}_{\text{silent}} \times {θ}_{\text{silent}} \text{;}$$

$${\text{I}}_{\text{amide}} \approx {\text{M}}_{\text{amide}} \times {θ}_{\text{amide}} \text{;}$$

As a result, Mₛᵢₗₑₙₜ⁻¹× Isilent ≈ Msilent⁻¹ ×Mₛᵢₗₑₙₜ ×Θₛᵢₗₑₙₜ, which means the decomposed contribution of each component Θₛᵢₗₑₙₜ ≈Mₛᵢₗₑₙₜ⁻¹× I_{silent.} Similarly, Θ_{amide} ≈M_{amide}⁻¹× I_{amide.} The matrix computation was carried out with MATLAB. To verify the linear unmixing model is satisfactory for multiparameter Raman spectral decomposition and robust to measurement noise, the single-cell Raman spectrum acquired was reconstructed with the calculated Θ. The Pearson correlation coefficient was employed to represent the similarity between reconstructed and ground spectrum and assess the robustness of the unmixing model.

### EXEMPLARY MATERIAL AND METHODS

### Exemplary Automatic Confocal Micro-Raman Spectrometer

As indicated herein above, a schematic diagram of the exemplary confocal Raman microscope according to an exemplary embodiment of the present disclosure is shown in FIG. 1A. For example, the exemplary single-cell Raman spectroscopy objective was underfilled and the illumination spot had a diameter of approximately 8 um as the result. In order to automatically acquire spectra, a brightfield image was taken and the cells in the images were identified and their locations were subsequently calculated. (See, e.g., FIGS. 5, 6A, and 6B). A motorized stage moved the sample slide so that the center of a cell aligned with the illumination spot. The process above was automated by a home-built software coded with Labview (e.g., National Instruments).

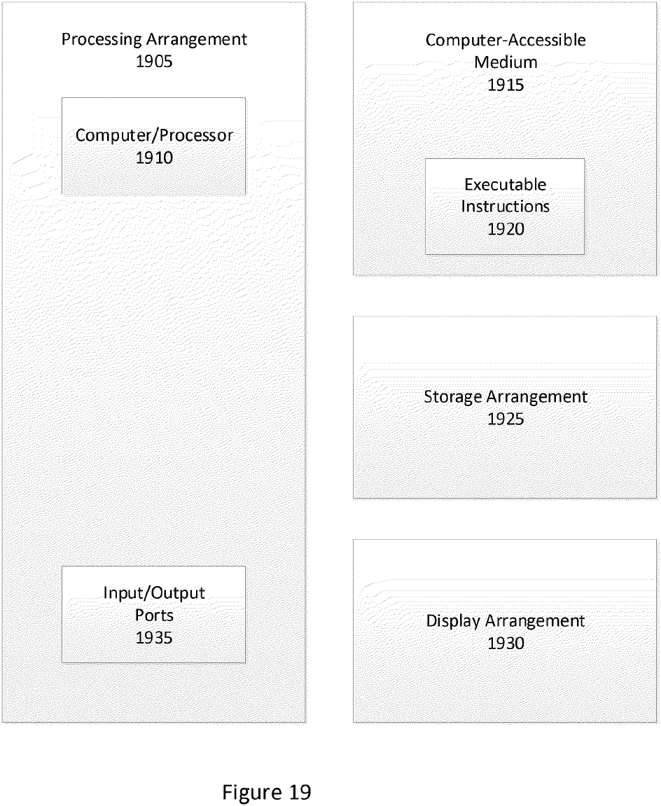

FIG. 19 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1905. Such processing/computing arrangement 1905 can be, for example entirely or a part of, or include, but not limited to, a computer/processor 1910 that can include, for example one or more microprocessors, and use instructions stored on a computer-accessible medium (e.g., RAM, ROM, hard drive, or other storage device).

As shown in FIG. 19, for example a computer-accessible medium 1915 (e.g., as described herein above, a storage device such as a hard disk, floppy disk, memory stick, CD-ROM, RAM, ROM, etc., or a collection thereof) can be provided (e.g., in communication with the processing arrangement 1905). The computer-accessible medium 1915 can contain executable instructions 1920 thereon. In addition or alternatively, a storage arrangement 1925 can be provided separately from the computer-accessible medium 1915, which can provide the instructions to the processing arrangement 1905 so as to configure the processing arrangement to execute certain exemplary procedures, processes, and methods, as described herein above, for example.

Further, the exemplary processing arrangement 1905 can be provided with or include an input/output ports 1935, which can include, for example a wired network, a wireless network, the internet, an intranet, a data collection probe, a sensor, etc. As shown in FIG. 19, the exemplary processing arrangement 1905 can be in communication with an exemplary display arrangement 1930, which, according to certain exemplary embodiments of the present disclosure, can be a touch-screen configured for inputting information to the processing arrangement in addition to outputting information from the processing arrangement, for example. Further, the exemplary display arrangement 1930 and/or a storage arrangement 1925 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties: 1. C. Dino Di, P. L. Luke, Dynamic Single-Cell Analysis for Quantitative Biology. Analytical Chemistry 78, 7918-7925 (2006).

2. Y. Zhao, E. B. Butler, M. Tan, Targeting cellular metabolism to improve cancer therapeutics. Cell Death Dis 4, e532 (2013).

3. M. Robertson-Tessi, R. J. Gillies, R. A. Gatenby, A. R. Anderson, Impact of metabolic heterogeneity on tumor growth, invasion, and treatment outcomes. Cancer Res 75, 1567-1579 (2015).

4. F. Yan, J. M. Timothy, B. Andreas, W. Y. Daniel, A. T. John, Multi-parameter phenotypic profiling: using cellular effects to characterize small-molecule compounds. Nat Rev Drug Discov 8, nrd2876 (2009).

5. L. D. Joanna et al., Probing the action of a novel anti-leukaemic drug therapy at the single cell level using modern vibrational spectroscopy techniques. Scientific Reports 7, 2649 (2017).

6. L. Lit-Hsin, F. W. Lani, J. A. Steven, Image-based multivariate profiling of drug responses from single cells. Nature Methods 4, nmeth1032 (2007).

7. O. K. Peter, P. N. Garry, Fluorescent cell barcoding in flow cytometry allows high-throughput drug screening and signaling profiling. Nature Methods 3, nmeth872 (2006).

8. E. P. Zachary et al., Multidimensional Drug Profiling By Automated Microscopy. Science 306, 1194-1198 (2004).

9. R. Subinoy et al., A multichannel nanosensor for instantaneous readout of cancer drug mechanisms. Nat Nanotechnol 10, 65-69 (2015).

10. S. S. Anthony et al., Single Cell Optical Imaging and Spectroscopy. Chem Rev 113, 2469-2527 (2013).

11. I. Georgakoudi, K. P. Quinn, Optical imaging using endogenous contrast to assess metabolic state. Annu Rev Biomed Eng 14, 351-367 (2012).

12. W. E. Joachim, Label-Free Monitoring of Cells in vitro (Springer, 2019).

13. Z. Zhao, Y. Shen, F. Hu, W. Min, Applications of vibrational tags in biological imaging by Raman microscopy. Analyst 142, 4018-4029 (2017).

14. Y. Hiroyuki et al., Alkyne-Tag Raman Imaging for Visualization of Mobile Small Molecules in Live Cells. Journal of the American Chemical Society 134, 20681-20689 (2012).

15. Y. Hiroyuki et al., Imaging of EdU, an Alkyne-Tagged Cell Proliferation Probe, by Raman Microscopy. Journal of the American Chemical Society 133, 6102-6105 (2011).

16. Y. Kim, S. Lee, H. Chung, H. Choi, K. Cha, Improving Raman spectroscopic differentiation of the geographical origin of rice by simultaneous illumination over a wide sample area. Journal of Raman Spectroscopy 40, 191-196 (2009).

17. P. Nicolas, I. S. Nicholas, Maximizing throughput in label-free microspectroscopy with hybrid Raman imaging. J Biomed Opt 20, 016007-016007 (2015).

18. W. S. Iwan, K. Roman, K. Christoph, P. Jürgen, Rapid acquisition of mean Raman spectra of eukaryotic cells for a robust single cell classification. Analyst 141, 6387-6395 (2016).

19. W. S. Iwan et al., High-Throughput Screening Raman Spectroscopy Platform for Label-Free Cellomics. Analytical Chemistry 90, 2023-2030 (2018).

20. S. Uwe, Metabolic networks in motion: 13C-based flux analysis. Mol Syst Biol 2, 62 (2006).

21. Z. Nicola, F. Sarah-Maria, R. Martin, S. Uwe, 13C-based metabolic flux analysis. Nat Protoc 4, 878-892 (2009).

22. D. Berry et al., Tracking heavy water (D2O) incorporation for identifying and sorting active microbial cells. Proc Natl Acad Sci U S A 112, E194-203 (2015).

23. W. Lu et al., Imaging Complex Protein Metabolism in Live Organisms by Stimulated Raman Scattering Microscopy with Isotope Labeling. ACS Chemical Biology 10, 901-908 (2015).

24. J. A. Steven, F. W. Lani, Cellular Heterogeneity: Do Differences Make a Difference? Cell 141, 559-563 (2010).

25. C. Krafft et al., Label-Free Molecular Imaging of Biological Cells and Tissues by Linear and Nonlinear Raman Spectroscopic Approaches. Angew Chem Int Ed Engl 56, 4392-4430 (2017).

26. J. Jaumot, A. de Juan, R. Tauler, MCR-ALS GUI 2.0: New features and applications. Chemometrics and Intelligent Laboratory Systems 140, 1-12 (2015).

27. K. Neymeyr, M. Sawall, D. Hess, Pure component spectral recovery and constrained matrix factorizations: concepts and applications. J Chemometr 24, n/a-n/a (2010).

28. F. Judith et al., Vibrational spectroscopic image analysis of biological material using multivariate curve resolution-alternating least squares (MCR-ALS). Nat Protoc 10, 217-240 (2015).

29. M. C. Fishman, J. A. Porter, Pharmaceuticals: a new grammar for drug discovery. Nature 437, 491-493 (2005).

30. H. Tomoda, K. Igarashi, J. C. Cyong, S. Omura, Evidence for an essential role of long chain acyl-CoA synthetase in animal cell proliferation. Inhibition of long chain acyl-CoA synthetase by triacsins caused inhibition of Raji cell proliferation. J Biol Chem 266, 4214-4219 (1991).

31. L. L. Baumbach, F. Marashi, M. Plumb, G. Stein, J. Stein, Inhibition of DNA replication coordinately reduces cellular levels of core and H1 histone mRNAs: requirement for protein synthesis. Biochemistry 23, 1618-1625 (1984).

32. P. Hai et al., High-throughput, label-free, single-cell photoacoustic microscopy of intratumoral metabolic heterogeneity. Nat Biomed Eng 3, 381-391 (2019).

33. L. H. Deborah, S. Valerie, Choosing the right cell line for breast cancer research. Breast Cancer Research 13, 215 (2011).

34. K. Christoph, P. Jürgen, The many facets of Raman spectroscopy for biomedical analysis. Anal Bioanal Chem 407, 699-717 (2015).

35. S. Stöckel, J. Kirchhoff, U. Neugebauer, P. Rösch, J. Popp, The application of Raman spectroscopy for the detection and identification of microorganisms. Journal of Raman Spectroscopy 47, 89-109 (2016).

36. R. Kiselev, I. W. Schie, S. Askrabic, C. Krafft, J. Popp, Design and first applications of a flexible Raman micro-spectroscopic system for biological imaging. Biomedical Spectroscopy and Imaging 5, 115-127 (2016).

37. S. Yuta et al., Label-free chemical imaging flow cytometry by high-speed multicolor stimulated Raman scattering. Proceedings of the National Academy of Sciences 116, 15842-15848 (2019).

38. S. Yizhi, Y. Huabing, E. H. Wei, Raman activated cell sorting. Curr Opin Chem Biol 33, 1-8 (2016).

39. L. Jinchao et al., Deep convolutional neural networks for Raman spectrum recognition: a unified solution. 142, 4067-4074 (2017).

40. M. J. Anderson, A new method for non-parametric multivariate analysis of variance. Austral Ecol 26, 32-46 (2001).

41. C. Beleites, U. Neugebauer, T. Bocklitz, C. Krafft, J. Popp, Sample size planning for classification models. Anal Chim Acta 760, 25-33 (2013).

42. N. Pavillon, A. J. Hobro, S. Akira, N. I. Smith, Noninvasive detection of macrophage activation with single-cell resolution through machine learning. Proc Natl Acad Sci U S A 115, E2676-E2685 (2018).

43. A. Germond et al., Cell type discrimination based on image features of molecular component distribution. Sci Rep 8, 11726 (2018).

44. S. H. Abigail et al., Diagnosing breast cancer by using Raman spectroscopy. P Natl Acad Sci Usa 102, 12371-12376 (2005).

45. P. Santosh Kumar et al., Label-Free Raman Spectroscopy Detects Stromal Adaptations in Premetastatic Lungs Primed by Breast Cancer. Cancer Res 77, 247-256 (2017).

46. A. C. S. Talari, C. A. Evans, I. Holen, R. E. Coleman, I. U. Rehman, Raman spectroscopic analysis differentiates between breast cancer cell lines. Journal of Raman Spectroscopy 46, 421-427 (2015).

47. N. Jens, S. Konstantin, H. Elmar, Metabolic flux analysis in eukaryotes. Curr Opin Biotech 21, 63-69 (2010).

48. R. S. Alan, C. R. Kahn, Insulin signalling and the regulation of glucose and lipid metabolism. Nature 414, 799-806 (2001).

49. Z. Yinan et al., Coordinated regulation of protein synthesis and degradation by mTORC1. Nature 513, 440-443 (2014).

50. C. Zhang et al., Stimulated Raman scattering flow cytometry for label-free single-particle analysis. Optica 4, 103 (2017).

51. H. Kotaro et al., High-throughput label-free molecular fingerprinting flow cytometry. Sci Adv 5, eaau0241 (2019).

52. W. E. Joachim, Label-Free Monitoring of Cells in vitro (Springer, 2019).

53. K. Christoph, P. Jürgen, The many facets of Raman spectroscopy for biomedical analysis. Anal Bioanal Chem 407, 699-717 (2015).

54. C. Krafft et al., Label-Free Molecular Imaging of Biological Cells and Tissues by Linear and Nonlinear Raman Spectroscopic Approaches. Angew Chem Int Ed Engl 56, 4392-4430 (2017).

55. S. Stöckel, J. Kirchhoff, U. Neugebauer, P. Rösch, J. Popp, The application of Raman spectroscopy for the detection and identification of microorganisms. Journal of Raman Spectroscopy 47, 89-109 (2016).

56. R. Kiselev, I. W. Schie, S. Askrabic, C. Krafft, J. Popp, Design and first applications of a flexible Raman micro-spectroscopic system for biological imaging. Biomedical Spectroscopy and Imaging 5, 115-127 (2016).

57. S. Yuta et al., Label-free chemical imaging flow cytometry by high-speed multicolor stimulated Raman scattering. Proceedings of the National Academy of Sciences 116, 15842-15848 (2019).

58. S. Yizhi, Y. Huabing, Er. H. Wei, Raman activated cell sorting. Curr Opin Chem Biol 33, 1-8 (2016).

59. L. Jinchao et al., Deep convolutional neural networks for Raman spectrum recognition: a unified solution. 142, 4067-4074 (2017).

30. C. Beleites, U. Neugebauer, T. Bocklitz, C. Krafft, J. Popp, Sample size planning for classification models. Anal Chim Acta 760, 25-33 (2013).

61. N. Pavillon, A. J. Hobro, S. Akira, N. I. Smith, Noninvasive detection of macrophage activation with single-cell resolution through machine learning. Proc Natl Acad Sci U S A 115, E2676-E2685 (2018).

62. M. J. Anderson, A new method for non-parametric multivariate analysis of variance. Austral Ecol 26, 32-46 (2001).

63. A. Germond et al., Cell type discrimination based on image features of molecular component distribution. Sci Rep 8, 11726 (2018).

64. T. Bocklitz, A. Walter, K. Hartmann, P. Rosch, J. Popp, How to pre-process Raman spectra for reliable and stable models? Anal Chim Acta 704, 47-56 (2011).

65. P. Lasch, Spectral pre-processing for biomedical vibrational spectroscopy and microspectroscopic imaging. Chemometrics and Intelligent Laboratory Systems 117, 100-114 (2012).

66. B. D. Beier, A. J. Berger, Method for automated background subtraction from Raman spectra containing known contaminants. Analyst 134, 1198-1202 (2009).

67. V. Mazet, C. Carteret, D. Brie, J. Idier, B. Humbert, Background removal from spectra by designing and minimising a non-quadratic cost function. Chemometrics and Intelligent Laboratory Systems 76, 121-133 (2005).

The following references are also hereby incorporated by reference in their entireties:

A1. Lin, J. R.; Izar, B.; Wang, S.; Yapp, C.; Mei, S.; Shah, P. M.; Santagata, S.; Sorger, P. K., Highly multiplexed immunofluorescence imaging of human tissues and tumors using t-CyCIF and conventional optical microscopes. Elife 2018, 7.

A2. Fan, Y.; Wang, S.; Zhang, F., Optical Multiplexed Bioassays for Improved Biomedical Diagnostics. Angew Chem Int Ed Engl 2019, 58 (38), 13208-13219.

A3. Cohen, L.; Walt, D. R., Highly Sensitive and Multiplexed Protein Measurements. Chem Rev 2019, 119 (1), 293-321.

A4. Bendall, S. C.; Nolan, G. P., From single cells to deep phenotypes in cancer. Nat Biotechnol 2012, 30 (7), 639-47.

A5. Perfetto, S. P., Pratip K. Chattopadhyay, and Mario Roederer, Seventeen-colour flow cytometry: unravelling the immune system. Nature Reviews Immunology 2004, 4 (8), 648-655.

A6. Robinson, J. P., and Mario Roederer, Flow cytometry strikes gold. Science 2015, 350 (6262), 739-740.

A7. de Vries, N. L.; Mahfouz, A.; Koning, F.; de Miranda, N., Unraveling the Complexity of the Cancer Microenvironment With Multidimensional Genomic and Cytometric Technologies. Front Oncol 2020, 10, 1254.

A8. Du, J.; Su, Y.; Qian, C.; Yuan, D.; Miao, K.; Lee, D.; Ng, A. H. C.; Wijker, R. S.; Ribas, A.; Levine, R. D.; Heath, J. R.; Wei, L., Raman-guided subcellular pharmaco-metabolomics for metastatic melanoma cells. Nature Communications 2020, 11 (1).

A9. Wei, L.; Hu, F.; Chen, Z.; Shen, Y.; Zhang, L.; Min, W., Live-Cell Bioorthogonal Chemical Imaging: Stimulated Raman Scattering Microscopy of Vibrational Probes. Acc Chem Res 2016, 49 (8), 1494-502.

A10. Huang, K. C.; Li, J.; Zhang, C.; Tan, Y.; Cheng, J. X., Multiplex Stimulated Raman Scattering Imaging Cytometry Reveals Lipid-Rich Protrusions in Cancer Cells under Stress Condition. iScience 2020, 23 (3), 100953.

A11. Hiramatsu, K., Takuro Ideguchi, Yusuke Yonamine, SangWook Lee, Yizhi Luo, Kazuki Hashimoto, Takuro Ito et al, High-throughput label-free molecular fingerprinting flow cytometry. Science advances 2019, 5 (1), eaau0241.

A12. Miao, Y.; Shi, L.; Hu, F.; Min, W., Probe design for super-multiplexed vibrational imaging. Phys Biol 2019, 16 (4), 041003.

A13. Dougan, J. A.; Faulds, K., Surface enhanced Raman scattering for multiplexed detection. Analyst 2012, 137 (3), 545-54.

A14. Wang, J.; Liang, D.; Feng, J.; Tang, X., Multicolor Cocktail for Breast Cancer Multiplex Phenotype Targeting and Diagnosis Using Bioorthogonal Surface-Enhanced Raman Scattering Nanoprobes. Anal Chem 2019, 91 (17), 11045-11054.

A15. Zavaleta, C. L., Bryan R. Smith, Ian Walton, William Doering, Glenn Davis, Borzoyeh Shojaei, Michael J. Natan, and Sanjiv S. Gambhir, Multiplexed imaging of surface enhanced Raman scattering nanotags in living mice using noninvasive Raman spectroscopy. Proceedings of the National Academy of Sciences 2009, 106 (32), 13511-13516.

A16. Shen, Y.; Hu, F.; Min, W., Raman Imaging of Small Biomolecules. Annu Rev Biophys 2019, 48, 347-369.

A17. Suzuki, Y.; Kobayashi, K.; Wakisaka, Y.; Deng, D.; Tanaka, S.; Huang, C. J.; Lei, C.; Sun, C. W.; Liu, H.; Fujiwaki, Y.; Lee, S.; Isozaki, A.; Kasai, Y.; Hayakawa, T.; Sakuma, S.; Arai, F.; Koizumi, K.; Tezuka, H.; Inaba, M.; Hiraki, K.; Ito, T.; Hase, M.; Matsusaka, S.; Shiba, K.; Suga, K.; Nishikawa, M.; Jona, M.; Yatomi, Y.; Yalikun, Y.; Tanaka, Y.; Sugimura, T.; Nitta, N.; Goda, K.; Ozeki, Y., Label-free chemical imaging flow cytometry by high-speed multicolor stimulated Raman scattering. Proc Natl Acad Sci U SA 2019, 116 (32), 15842-15848.

A18. Zhang, Q.; Zhang, P.; Gou, H.; Mou, C.; Huang, W. E.; Yang, M.; Xu, J.; Ma, B., Towards high-throughput microfluidic Raman-activated cell sorting. Analyst 2015, 140 (18), 6163-74.

A19. Hiramatsu, K.; Yamada, K.; Lindley, M.; Suzuki, K.; Goda, K., Large-scale label-free single-cell analysis of paramylon in Euglena gracilis by high-throughput broadband Raman flow cytometry. Biomed Opt Express 2020, 11 (4), 1752-1759.

A20. Verma, R. S.; Ahlawat, S.; Uppal, A., Optical guiding-based cell focusing for Raman flow cell cytometer. Analyst 2018, 143 (11), 2648-2655.

A21. Bendall, S. C., Erin F. Simonds, Peng Qiu, D. Amir El-ad, Peter O. Krutzik, Rachel Finck, Robert V. Bruggner et al, Single-cell mass cytometry of differential immune and drug responses across a human hematopoietic continuum. Science 2011, 332 (6030), 687-696. A22. Spitzer, M. H.; Nolan, G. P., Mass Cytometry: Single Cells, Many Features. Cell 2016, 165 (4), 780-91.

A23. Wu, X.; DeGottardi, Q.; Wu, I. C.; Yu, J.; Wu, L.; Ye, F.; Kuo, C. T.; Kwok, W. W.; Chiu, D. T., Lanthanide-Coordinated Semiconducting Polymer Dots Used for Flow Cytometry and Mass Cytometry. Angew Chem Int Ed Engl 2017, 56 (47), 14908-14912.

A24. Baumgarth, N., and Mario Roederer, A practical approach to multicolor flow cytometry for immunophenotyping. Journal of immunological methods 2000, 243 (1-2), 77-97.

A25. Hu, F.; Zeng, C.; Long, R.; Miao, Y.; Wei, L.; Xu, Q.; Min, W., Supermultiplexed optical imaging and barcoding with engineered polyynes. Nat Methods 2018, 15 (3), 194-200.

A26. Wei, L.; Chen, Z.; Shi, L.; Long, R.; Anzalone, A. V.; Zhang, L.; Hu, F.; Yuste, R.; Cornish, V. W.; Min, W., Super-multiplex vibrational imaging. Nature 2017, 544 (7651), 465-470.

A27. Hu, F.; Brucks, S. D.; Lambert, T. H.; Campos, L. M.; Min, W., Stimulated Raman scattering of polymer nanoparticles for multiplexed live-cell imaging. Chem Commun (Camb) 2017, 53 (46), 6187-6190.

A28. Li, S.; Chen, T.; Wang, Y.; Liu, L.; Lv, F.; Li, Z.; Huang, Y.; Schanze, K. S.; Wang, S., Conjugated Polymer with Intrinsic Alkyne Units for Synergistically Enhanced Raman Imaging in Living Cells. Angew Chem Int Ed Engl 2017, 56 (43), 13455-13458.

A29. Zavaleta, C. L., Bryan R. Smith, Ian Walton, William Doering, Glenn Davis, Borzoyeh Shojaei, Michael J. Natan, and Sanjiv S. Gambhir, Multiplexed imaging of surface enhanced Raman scattering nanotags in living mice using noninvasive Raman spectroscopy. Proceedings of the National Academy of Sciences 2009, 106 (32), 13511-13516.

A30. Zhang, Y.; Gu, Y.; He, J.; Thackray, B. D.; Ye, J., Ultrabright gap-enhanced Raman tags for high-speed bioimaging. Nat Commun 2019, 10 (1), 3905.

A31. Langer, J.; Jimenez de Aberasturi, D.; Aizpurua, J.; Alvarez-Puebla, R. A.; Auguie, B.; Baumberg, J. J.; Bazan, G. C.; Bell, S. E. J.; Boisen, A.; Brolo, A. G.; Choo, J.; Cialla-May, D.; Deckert, V.; Fabris, L.; Faulds, K.; Garcia de Abajo, F. J.; Goodacre, R.; Graham, D.; Haes, A. J.; Haynes, C. L.; Huck, C.; Itoh, T.; Kall, M.; Kneipp, J.; Kotov, N. A.; Kuang, H.; Le Ru, E. C.; Lee, H. K.; Li, J. F.; Ling, X. Y.; Maier, S. A.; Mayerhofer, T.; Moskovits, M.; Murakoshi, K.; Nam, J. M.; Nie, S.; Ozaki, Y.; Pastoriza-Santos, I.; Perez-Juste, J.; Popp, J.; Pucci, A.; Reich, S.; Ren, B.; Schatz, G. C.; Shegai, T.; Schlucker, S.; Tay, L. L.; Thomas, K. G.; Tian, Z. Q.; Van Duyne, R. P.; Vo-Dinh, T.; Wang, Y.; Willets, K. A.; Xu, C.; Xu, H.; Xu, Y.; Yamamoto, Y. S.; Zhao, B.; Liz-Marzan, L. M., Present and Future of Surface-Enhanced Raman Scattering. ACS Nano 2020, 14 (1), 28-117.

A32. Zhao, Z. C., C.; Wei, S.; Xiong, H.; Hu, F.; Miao, Y.; Jin, T. and Min, W.*, Ultra-bright Raman dots (Rdots) for multiplexed optical imaging. Nature Communications (in press).

A33. Wei, Y.; Deng, X.; Xie, Z.; Cai, X.; Liang, S.; Ma, P. a.; Hou, Z.; Cheng, Z.; Lin, J., Enhancing the Stability of Perovskite Quantum Dots by Encapsulation in Crosslinked Polystyrene Beads via a Swelling-Shrinking Strategy toward Superior Water Resistance. Advanced Functional Materials 2017, 27 (39).

A34. Zhao, Z.; Shen, Y.; Hu, F.; Min, W., Applications of vibrational tags in biological imaging by Raman microscopy. Analyst 2017, 142 (21), 4018-4029.

A35. Yamakoshi, H.; Dodo, K.; Palonpon, A.; Ando, J.; Fujita, K.; Kawata, S.; Sodeoka, M., Alkyne-tag Raman imaging for visualization of mobile small molecules in live cells. J Am Chem Soc 2012, 134 (51), 20681-9.

A36. Tian, S.; Li, H.; Li, Z.; Tang, H.; Yin, M.; Chen, Y.; Wang, S.; Gao, Y.; Yang, X.; Meng, F.; Lauher, J. W.; Wang, P.; Luo, L., Polydiacetylene-based ultrastrong bioorthogonal Raman probes for targeted live-cell Raman imaging. Nat Commun 2020, 11 (1), 81.

A37. Bentzen, E. L., Ian D. Tomlinson, John Mason, Paul Gresch, Michael R. Warnement, David Wright, Elaine Sanders-Bush, Randy Blakely, and Sandra J. Rosenthal, Surface modification to reduce nonspecific binding of quantum dots in live cell assays. Bioconjugate chemistry 2005, 16 (6), 1488-1494.

A38. Zhao, Z.; Chen, C.; Xiong, H.; Ji, J.; Min, W., Metabolic Activity Phenotyping of Single Cells with Multiplexed Vibrational Probes. Anal Chem 2020, 92 (14), 9603-9612.

A39. Ghosh, S. C.; Neslihan Alpay, S.; Klostergaard, J., CD44: a validated target for improved delivery of cancer therapeutics. Expert Opin Ther Targets 2012, 16 (7), 635-50.

A40. Soenen, S. J.; Himmelreich, U.; Nuytten, N.; Pisanic, T. R., 2nd; Ferrari, A.; De Cuyper, M., Intracellular nanoparticle coating stability determines nanoparticle diagnostics efficacy and cell functionality. Small 2010, 6 (19), 2136-45.

A41. Collinet, C.; Stöter, M.; Bradshaw, C. R.; Samusik, N.; Rink, J. C.; Kenski, D.; Habermann, B.; Buchholz, F.; Henschel, R.; Mueller, M. S.; Nagel, W. E.; Fava, E.; Kalaidzidis, Y.; Zerial, M., Systems survey of endocytosis by multiparametric image analysis. Nature 2010, 464 (7286), 243-249.

A42. Mellman, I.; Yarden, Y., Endocytosis and cancer. Cold Spring Harb Perspect Biol 2013, 5 (12), a016949.

A43. Rejman, J., Volker Oberle, Inge S. Zuhorn, and Dick Hoekstra, Size-dependent internalization of particles via the pathways of clathrinand caveolae-mediated endocytosis. Biochemical Journal 2004, 377 (1), 159-169.

A44. Vercauteren, D.; Vandenbroucke, R. E.; Jones, A. T.; Rejman, J.; Demeester, J.; De Smedt, S. C.; Sanders, N. N.; Braeckmans, K., The use of inhibitors to study endocytic pathways of gene carriers: optimization and pitfalls. Mol Ther 2010, 18 (3), 561-9.

A45. Yang, Z.; Wu, F.; Yang, H.; Zhou, P., Endocytosis mechanism of a novel proteoglycan, extracted from Ganoderma lucidum, in HepG2 cells. RSC Advances 2017, 7 (66), 41779-41786.

A46. Jeng, R. L., and Matthew D. Welch, Cytoskeleton: actin and endocytosis-no longer the weakest link. Current Biology 2001, 11 (17), R691-R694.

A47. Wu, M.; Guo, H.; Liu, L.; Liu, Y.; Xie, L., Size-dependent cellular uptake and localization profiles of silver nanoparticles. Int J Nanomedicine 2019, 14, 4247-4259.

A48. Wang, T.; Wang, L.; Li, X.; Hu, X.; Han, Y.; Luo, Y.; Wang, Z.; Li, Q.; Aldalbahi, A.; Wang, L.; Song, S.; Fan, C.; Zhao, Y.; Wang, M.; Chen, N., Size-Dependent Regulation of Intracellular Trafficking of Polystyrene Nanoparticle-Based Drug-Delivery Systems. ACS Appl Mater Interfaces 2017, 9 (22), 18619-18625.

A49. Rejman, J., Volker Oberle, Inge S. Zuhorn, and Dick Hoekstra, Size-dependent internalization of particles via the pathways of clathrin-and caveolae-mediated endocytosis. Biochemical Journal 2004, 377 (1), 159-169.

A50. Maaten, L. v. d., and Geoffrey Hinton, Visualizing data using t-SNE. Journal of machine learning research 2008, 9 (11), 2579-2605.

A51. Muhamadali, H.; Chisanga, M.; Subaihi, A.; Goodacre, R., Combining Raman and FT-IR spectroscopy with quantitative isotopic labeling for differentiation of E. coli cells at community and single cell levels. Anal Chem 2015, 87 (8), 4578-86.

A52. Wei, L., Fanghao Hu, Yihui Shen, Zhixing Chen, Yong Yu, Chih-Chun Lin, Meng C. Wang, and Wei Min, Live-cell imaging of alkyne-tagged small biomolecules by stimulated Raman scattering. Nature methods 2014, 11 (4), 410-412.

A53. Chen, Z.; Paley, D. W.; Wei, L.; Weisman, A. L.; Friesner, R. A.; Nuckolls, C.; Min, W., Multicolor live-cell chemical imaging by isotopically edited alkyne vibrational palette. J Am Chem Soc 2014, 136 (22), 8027-33.

A54. Zhang, L.; Shi, L.; Shen, Y.; Miao, Y.; Wei, M.; Qian, N.; Liu, Y.; Min, W., Spectral tracing of deuterium for imaging glucose metabolism. Nat Biomed Eng 2019, 3 (5), 402-413.

A55. Shi, L.; Zheng, C.; Shen, Y.; Chen, Z.; Silveira, E. S.; Zhang, L.; Wei, M.; Liu, C.; de Sena-Tomas, C.; Targoff, K.; Min, W., Optical imaging of metabolic dynamics in animals. Nat Commun 2018, 9 (1), 2995.

A56. Yaghoobi, M.; Wu, D.; Clewes, R. J.; Davies, M. E.; Burgess, D.; Owen, G.; Bouma, H.; Carlysle-Davies, F.; Stokes, R. J.; Yitzhaky, Y., Fast sparse Raman spectral unmixing for chemical fingerprinting and quantification. In Optics and Photonics for Counterterrorism, Crime Fighting, and Defence XII, 2016.

A57. Feng, Y.; Mitchison, T. J.; Bender, A.; Young, D. W.; Tallarico, J. A., Multi-parameter phenotypic profiling: using cellular effects to characterize small-molecule compounds. Nat Rev Drug Discov 2009, 8 (7), 567-78.

A58. Perlman, Z. E., Michael D. Slack, Yan Feng, Timothy J. Mitchison, Lani F. Wu, and Steven J. Altschuler, Multidimensional drug profiling by automated microscopy. Science 2004, 306 (5699), 1194-1198.

A59. Leonavicius, K.; Nainys, J.; Kuciauskas, D.; Mazutis, L., Multi-omics at single-cell resolution: comparison of experimental and data fusion approaches. Curr Opin Biotechnol 2019, 55, 159-166.

A60. Zhu, C.; Preissl, S.; Ren, B., Single-cell multimodal omics: the power of many. Nat Methods 2020, 17 (1), 11-14.

A61. Nahta, R.; Esteva, F. J., Trastuzumab: triumphs and tribulations. Oncogene 2007, 26 (25), 3637-43.

A62. Gough, D. R.; Cotter, T. G., Hydrogen peroxide: a Jekyll and Hyde signalling molecule. Cell Death Dis 2011, 2, e213.

A63. Park, Y.; Koga, Y.; Su, C.; Waterbury, A. L.; Johnny, C. L.; Liau, B. B., Versatile Synthetic Route to Cycloheximide and Analogues That Potently Inhibit Translation Elongation. Angew Chem Int Ed Engl 2019, 58 (16), 5387-5391.

A64. Diring, J.; Mouilleron, S.; McDonald, N. Q.; Treisman, R., RPEL-family rhoGAPs link Rac/Cdc42 GTP loading to G-actin availability. Nat Cell Biol 2019, 21 (7), 845-855. A65. Brown, A., Sanjay Kumar, and Paul B. Tchounwou, Cisplatin-based chemotherapy of human cancers. Journal of cancer science & therapy 2019, 11 (4).

A66. Parisis, N.; Krasinska, L.; Harker, B.; Urbach, S.; Rossignol, M.; Camasses, A.; Dewar, J.; Morin, N.; Fisher, D., Initiation of DNA replication requires actin dynamics and formin activity. EMBO J 2017, 36 (21), 3212-3231.

A67. Ornelles, D. A., E. G. Fey, and S. Penman, Cytochalasin releases mRNA from the cytoskeletal framework and inhibits protein synthesis. Molecular and cellular biology 1986, 6 (5), 1650-1662.

A68. Samanna, V.; Ma, T.; Mak, T. W.; Rogers, M.; Chellaiah, M. A., Actin polymerization modulates CD44 surface expression, MMP-9 activation, and osteoclast function. J Cell Physiol 2007, 213 (3), 710-20.

A69. McMahon, D., Cycloheximide is not a specific inhibitor of protein synthesis in vivo.” Plant Physiology. Plant Physiology 1975, 55 (5), 815-821.

A70. Shen, Y.; Xu, F.; Wei, L.; Hu, F.; Min, W., Live-cell quantitative imaging of proteome degradation by stimulated Raman scattering. Angew Chem Int Ed Engl 2014, 53 (22), 5596-9.

A71. Asad, N. R., Lidia Maria Buarque Oliveira Asad, Carlos Eduardo Bonacossa de Almeida, Israel Felzenszwalb, Januário Bispo Cabral-Neto, and Alvaro Costa Leitão, Several pathways of hydrogen peroxide action that damage the E. coli genome. Genetics and Molecular Biology 2004, 27 (2), 291-303.

A72. Gorini, S.; De Angelis, A.; Berrino, L.; Malara, N.; Rosano, G.; Ferraro, E., Chemotherapeutic Drugs and Mitochondrial Dysfunction: Focus on Doxorubicin, Trastuzumab, and Sunitinib. Oxid Med Cell Longev 2018, 2018, 7582730.

A73. Wang, Y., Yingfang Zhu, Yu Ling, Haiyan Zhang, Peng Liu, František Baluška, Jozef Šamaj, Jinxing Lin, and Qinli Wang, Disruption of actin filaments induces mitochondrial Ca 2+ release to the cytoplasm and [Ca 2+] c changes in Arabidopsis root hairs. BMC plant biology 2010, 10 (1), 1-13.

A74. Varbiro, G., Balazs Veres, Ferenc Gallyas Jr, and Balazs Sumegi, Direct effect of Taxol on free radical formation and mitochondrial permeability transition. Free Radical Biology and Medicine 2001, 31 (4), 548-558.

A75. Tada-Oikawa, S.; Hiraku, Y.; Kawanishi, M.; Kawanishi, S., Mechanism for generation of hydrogen peroxide and change of mitochondrial membrane potential during rotenone-induced apoptosis. Life Sci 2003, 73 (25), 3277-88.

A76. Srivatsan, S. R., José L. McFaline-Figueroa, Vijay Ramani, Lauren Saunders, Junyue Cao, Jonathan Packer, Hannah A. Pliner et al, Massively multiplex chemical transcriptomics at single-cell resolution. Science 2020, 367 (6473), 45-51.

A77. McGinnis, C. S.; Patterson, D. M.; Winkler, J.; Conrad, D. N.; Hein, M. Y.; Srivastava, V.; Hu, J. L.; Murrow, L. M.; Weissman, J. S.; Werb, Z.; Chow, E. D.; Gartner, Z. J., MULTI-seq: sample multiplexing for single-cell RNA sequencing using lipid-tagged indices. Nat Methods 2019, 16 (7), 619-626.

A78. McFarland, J. M.; Paolella, B. R.; Warren, A.; Geiger-Schuller, K.; Shibue, T.; Rothberg, M.; Kuksenko, O.; Colgan, W. N.; Jones, A.; Chambers, E.; Dionne, D.; Bender, S.; Wolpin, B. M.; Ghandi, M.; Tirosh, I.; Rozenblatt-Rosen, O.; Roth, J. A.; Golub, T. R.; Regev, A.; Aguirre, A. J.; Vazquez, F.; Tsherniak, A., Multiplexed single-cell transcriptional response profiling to define cancer vulnerabilities and therapeutic mechanism of action. Nat Commun 2020, 11 (1), 4296.

A79. Sun, W.; Li, J.; Jiang, H. G.; Ge, L. P.; Wang, Y., Diagnostic value of MUC1 and EpCAM mRNA as tumor markers in differentiating benign from malignant pleural effusion. QJM 2014, 107 (12), 1001-7.

A80. Wee, P.; Wang, Z., Regulation of EGFR Endocytosis by CBL During Mitosis. Cells 2018, 7 (12).

A81. Jeong, H., Sean P. Mason, A-L. Barabási, and Zoltan N. Oltvai, Lethality and centrality in protein networks. Nature 2001, 411 (6833), 41-42.

A82. Santolini, M.; Barabasi, A. L., Predicting perturbation patterns from the topology of biological networks. Proc Natl Acad Sci U S A 2018, 115 (27), E6375-E6383.

A83. Csermely, P.; Korcsmaros, T.; Kiss, H. J.; London, G.; Nussinov, R., Structure and dynamics of molecular networks: a novel paradigm of drug discovery: a comprehensive review. Pharmacol Ther 2013, 138 (3), 333-408.

A84. Spiro, Z.; Kovacs, I. A.; Csermely, P., Drug-therapy networks and the prediction of novel drug targets. J Biol 2008, 7 (6), 20.

A85. Nitta, N.; Iino, T.; Isozaki, A.; Yamagishi, M.; Kitahama, Y.; Sakuma, S.; Suzuki, Y.; Tezuka, H.; Oikawa, M.; Arai, F.; Asai, T.; Deng, D.; Fukuzawa, H.; Hase, M.; Hasunuma, T.; Hayakawa, T.; Hiraki, K.; Hiramatsu, K.; Hoshino, Y.; Inaba, M.; Inoue, Y.; Ito, T.; Kajikawa, M.; Karakawa, H.; Kasai, Y.; Kato, Y.; Kobayashi, H.; Lei, C.; Matsusaka, S.; Mikami, H.; Nakagawa, A.; Numata, K.; Ota, T.; Sekiya, T.; Shiba, K.; Shirasaki, Y.; Suzuki, N.; Tanaka, S.; Ueno, S.; Watarai, H.; Yamano, T.; Yazawa, M.; Yonamine, Y.; Di Carlo, D.; Hosokawa, Y.; Uemura, S.; Sugimura, T.; Ozeki, Y.; Goda, K., Raman image-activated cell sorting. Nat Commun 2020, 11 (1), 3452.

A86. Nitta, N.; Sugimura, T.; Isozaki, A.; Mikami, H.; Hiraki, K.; Sakuma, S.; Iino, T.; Arai, F.; Endo, T.; Fujiwaki, Y.; Fukuzawa, H.; Hase, M.; Hayakawa, T.; Hiramatsu, K.; Hoshino, Y.; Inaba, M.; Ito, T.; Karakawa, H.; Kasai, Y.; Koizumi, K.; Lee, S.; Lei, C.; Li, M.; Maeno, T.; Matsusaka, S.; Murakami, D.; Nakagawa, A.; Oguchi, Y.; Oikawa, M.; Ota, T.; Shiba, K.; Shintaku, H.; Shirasaki, Y.; Suga, K.; Suzuki, Y.; Suzuki, N.; Tanaka, Y.; Tezuka, H.; Toyokawa, C.; Yalikun, Y.; Yamada, M.; Yamagishi, M.; Yamano, T.; Yasumoto, A.; Yatomi, Y.; Yazawa, M.; Di Carlo, D.; Hosokawa, Y.; Uemura, S.; Ozeki, Y.; Goda, K., Intelligent Image-Activated Cell Sorting. Cell 2018, 175 (1), 266-276 e13.

A87. Nabavinia, M. S.; Gholoobi, A.; Charbgoo, F.; Nabavinia, M.; Ramezani, M.; Abnous, K., Anti-MUC1 aptamer: A potential opportunity for cancer treatment. Med Res Rev 2017, 37 (6), 1518-1539.

A88. Labib, M.; Green, B.; Mohamadi, R. M.; Mepham, A.; Ahmed, S. U.; Mahmoudian, L.; Chang, I. H.; Sargent, E. H.; Kelley, S. O., Aptamer and Antisense-Mediated Two-Dimensional Isolation of Specific Cancer Cell Subpopulations. J Am Chem Soc 2016, 138 (8), 2476-9.

What is claimed is:

1. A non-transitory computer-accessible medium having stored thereon computer-executable instructions for determining phenotypic information for at least one cell, wherein, when a computing arrangement executes the instructions, the computing arrangement is configured to perform procedures comprising:
generating spectral information of the at least one cell using a Raman spectroscopy procedure that is based on at least one vibrational probe; and
determining the phenotypic information based on the spectral information.

2. The computer-accessible medium of claim 1, wherein the Raman spectroscopy procedure is a single-cell spontaneous Raman spectroscopy procedure.

3. The computer-accessible medium of claim 2, wherein the single-cell spontaneous Raman spectroscopy procedure is performed using a whole-cell confocal micro-Raman spectrometer.

4. The computer-accessible medium of claim 3, wherein the whole-cell confocal micro-Raman spectrometer has or provides an illumination spot size of one of (i) between about 1 µm to about 5 µm, (ii) between about 5 µm to about 10 µm, or (iii) between about 10 µm to about 100 µm.

5. The computer-accessible medium of claim 3, wherein the whole-cell confocal micro-Raman spectrometer has or provides a pinhole size of one of (i) between about 50 µm to about 200µ, (ii) between about 200 µm to about 400 µm, or (iii) between about 400 µm to about 1000 µm.

6. The computer-accessible medium of claim 1, wherein the at least one vibrational probe includes one of (i) at least one Deuterium-labeled branched-chain amino acid, or (ii) at least one deuterium-labeled palmitic acid.

7. The computer-accessible medium of claim 1, wherein the at least one vibrational probe includes or provides at least one metabolite molecule that is used by a living organism for biosynthesis.

8. The computer-accessible medium of claim 1, wherein the at least one vibrational probe includes at least one of (i) natural perdeuterated amino acids, (ii) partially deuterated amino acids, (iii) palmitic acid, (iv) oleic acid, (v) deuterated cholesterol, (vi) heavy water, (vii) deuterated glucose, (viii) deuterated acetate, (ix) alkyne bearing amino acids, (x) 1-homopropargylglycine, (xi) alkyne bearing fatty acids, (xii) 17-octadecynoic acid, (xiii) alkyne bearing nucleic acids, (xiv) 5-ethynyl-2′-deoxyuridine, (xv) 5-ethynyl uridine, (xvi) propargylcholine, (xvii) 3-O-propargyl-D-glucose, (xviii) Carbow orgenell dyes, (xix) Carbow-Mito, (xx) Carbow-Lyso, (xxi) Carbow-ER, (xxii) at least one drug, (xxiii) erlotinib, (xxiv) rhabduscin, (xxv) terbinafine, (xxvi) or carbonyl cyanide 4-(trifluoromethoxy)phenylhydrazone.

9. The computer-accessible medium of claim 1, wherein the at least one vibrational probe has or provides at least one Raman peak in a cell-silent spectral region.

10. The computer-accessible medium of claim 9, wherein the cell-silent spectral region is between 1800 cm-1 and 2800 cm-1.

11. The computer-accessible medium of claim 1, wherein the at least one vibrational probe includes at least one Raman-active nanoparticle.

12. The computer-accessible medium of claim 11, wherein the at least one Raman-active nanoparticle has a size of between 10 nanometer and 500 nanometer.

13. The computer-accessible medium of claim 11, wherein the at least one Raman-active nanoparticle has a Raman peak that is distinguishable from at least one of Raman peaks of the at least one cell.

14. A system for determining phenotypic information for at least one cell, comprising:
a computer hardware arrangement configured to:
generate spectral information of the at least one cell using a Raman spectroscopy procedure that is based on at least one vibrational probe; and
determine the phenotypic information based on the spectral information.

15. The system of claim 14, wherein the Raman spectroscopy procedure is a single-cell spontaneous Raman spectroscopy procedure.

16. The system of claim 15, wherein the single-cell spontaneous Raman spectroscopy procedure is performed using a whole-cell confocal micro-Raman spectrometer.

17. The system of claim 16, wherein the whole-cell confocal micro-Raman spectrometer has or provides an illumination spot size of one of (i) between about 1 µm to about 5 µm, (ii) between about 5 µm to about 10 µm, or (iii) between about 10 µm to about 100 µm.

18. The system of claim 16, wherein the whole-cell confocal micro-Raman spectrometer has or provides a pinhole size of one of (i) between about 50 µm to about 200µ, (ii) between about 200 µm to about 400 µm, or (iii) between about 400 µm to about 1000 µm.

19. The system of claim 14, wherein the at least one vibrational probe includes one of (i) at least one Deuterium-labeled branched-chain amino acid, or (ii) at least one deuterium-labeled palmitic acid.

20-26. (canceled)

27. A method for determining phenotypic information for at least one cell, comprising:
generating spectral information of the at least one cell using a Raman spectroscopy procedure that is based on at least one vibrational probe; and
using a computer hardware arrangement, determining the phenotypic information based on the spectral information.

28-39. (canceled)
